(19) United States
(12) Patent Application Publication
(10) Pub. No.: US 2024/0068017 A1
(43) Pub. Date: Feb. 29, 2024

(54) METHODS FOR ANALYTE CAPTURE DETERMINATION

(71) Applicant: 10x Genomics, Inc., Pleasanton, CA (US)

(72) Inventors: Joakim Lundeberg, Stockholm (SE); Eva Gracia Villacampa, Solna (SE)

(21) Appl. No.: 18/269,593

(22) PCT Filed: Dec. 28, 2021
(86) PCT No.: PCT/US2021/065340

§ 371 Date: Jun. 26, 2023

Related U.S. Application Data
(60) Provisional application No. 63/132,112, filed on Dec. 30, 2020.
(60) Provisional application No. 63/146,815, filed on Feb. 8, 2021.

(51) Int. Cl. C12Q 1/6837; G01N 1/30; G01N 21/64

(52) U.S. Cl. CPC C12Q 1/6837; G01N 1/30; G01N 21/6458; G01N 2001/302

(57) ABSTRACT

Provided herein are methods and kits for determining the optimal conditions for analyte capture in biological samples.

### CROSS-REFERENCE TO RELATED APPLICATIONS

This application claims priority to U.S. Provisional Patent Application No. 63/132,112, filed on Dec. 30, 2020 and U.S. Provisional Patent Application No. 63/146,815, filed on Feb. 8, 2021. The contents of these applications are incorporated herein by reference in their entireties.

### BACKGROUND

Cells within a tissue have differences in cell morphology and/or function due to varied analyte levels (e.g., gene and/or protein expression) within the different cells. The specific position of a cell within a tissue (e.g., the cell's position relative to neighboring cells or the cell's position relative to the tissue microenvironment) can affect, e.g., the cell's morphology, differentiation, fate, viability, proliferation, behavior, signaling, and cross-talk with other cells in the tissue.

Spatial heterogeneity has been previously studied using techniques that typically provide data for a handful of analytes in the context of intact tissue or a portion of a tissue (e.g., tissue section), or provide significant analyte data from individual, single cells, but fails to provide information regarding the position of the single cells from the originating biological sample (e.g., tissue).

Determining the optimal conditions for detecting analytes within a biological sample, including fixed biological samples, includes determining the optimal conditions for capturing analytes. Generally, captured analytes are reverse transcribed with labelled nucleotides which can then be detected under different conditions (e.g., permeabilization conditions). However, in some biological samples (e.g., fixed biological samples) analytes can be degraded and detection with labelled nucleotides can be challenging. Thus, there remains a need for methods to determine optimal conditions for analyte capture in biological samples, including fixed biological samples.

### SUMMARY

Determining the conditions for detecting the maximum amount of usable analytes within a biological sample (e.g., fixed biological samples) includes determining optimal conditions for capturing analytes. Generally, captured analytes are reverse transcribed with labelled nucleotides and are detected under different experimental conditions in order to determine the optimal conditions for performing spatial analysis. However, in some biological samples (e.g., fixed biological samples) analytes can be degraded and detection with labelled nucleotides can be challenging. Thus, to remove the dependency of incorporating labelled nucleotides during cDNA synthesis the present disclosure features methods of detecting the template switch oligonucleotide (TSO) with one or more labelled probes complementary to the TSO.

Provided herein are methods, including (a) a biological sample on an array including a plurality of capture probes, where a capture probe includes a capture domain that specifically binds to an analyte in the biological sample; (b) extending a 3′ end of the capture probes using the analyte as a template to generate an extended capture probe; (c) adding to the 3′ end of the extended capture probes a first homopolymeric sequence; (d) contacting the extended capture probes with a template switching oligonucleotide including a strand of nucleic acid including in a 5′ to 3′ direction: a primer binding sequence and a second homopolymeric sequence, where the second homopolymeric sequence binds specifically to the first homopolymeric sequence; (e) extending the 3′ end of the extended capture probes using the template switching oligonucleotide as a template; (0 removing the analyte and the template-switching oligonucleotides from the extended capture probes; (g) hybridizing one or more labeled probes including the template switching oligonucleotide primer binding sequence or a portion thereof to the extended capture probes; and (h) detecting the labeled probes hybridized to extended capture probes.

In some embodiments, steps (a)-(h) are performed under a first set of conditions, and the method includes repeating steps (a)-(h) under a second set of conditions.

In some embodiments, the method includes staining the biological sample. In some embodiments, the staining includes hematoxylin and eosin. In some embodiments, includes imaging the biological sample. In some embodiments, imaging includes light field, bright field, dark field, phase contrast, fluorescence microscopy, reflection, interference, and confocal microscopy.

In some embodiments, the capture probe of the plurality of capture probes includes a spatial barcode. In some embodiments, the capture probe of the plurality of capture probes includes one or more functional domains, a unique molecular identifier, a cleavage domain, or any combination thereof.

In some embodiments, a labeled probe of the one or more labeled probes includes a fluorescent label.

In some embodiments, the adding in step (c) is performed using a terminal deoxynucleotidyl transferase.

In some embodiments, the removing in step (f) includes one or more washing steps. In some embodiments, the one or more washing steps includes the use of an acidic buffer. In some embodiments, the acidic buffer includes KOH.

In some embodiments, the array includes one or more features. In some embodiments, the one or more features includes a bead, a well or a spot.

In some embodiments, the biological sample is a tissue sample. In some embodiments, the tissue sample is a tissue section. In some embodiments, the tissue sample is a fixed tissue sample. In some embodiments, the fixed tissue sample is a fixed tissue section. In some embodiments, the fixed tissue sample is formalin-fixed paraffin-embedded tissue section.

In some embodiments, the analyte is DNA. In some embodiments, the analyte is RNA. In some embodiments, the RNA is mRNA.

In some embodiments, the capture domain includes a poly(T) sequence. In some embodiments, the first homopolymeric sequence is a poly(C) sequence and the second homopolymeric sequence is a poly(G) sequence.

Also provided herein are methods including: (a) contacting a biological sample with an array including a plurality of capture probes, where a capture probe includes a capture domain that specifically binds to an analyte in the biological sample; (b) staining and imaging the biological sample; (c) extending a 3′ end of the capture probes using the analytes as a template to generate extended capture probes; (d) adding to the 3′ end of the extended capture probes a first homopolymeric sequence; (e) contacting the extended capture probes with a template switching oligonucleotide including in a 5′ to 3′ direction: a primer binding sequence and a second homopolymeric sequence, where the second homopolymeric sequence binds specifically to the first homopolymeric sequence; (0 extending the 3′ end of the extended capture probes using the template switching oligonucleotide as a template; (g) removing the analytes and the template-switching oligonucleotides from the extended capture probes; (h) hybridizing one or more labeled probes including the template switching oligonucleotide or a portion thereof to the extended capture probes; and (i) detecting the one or more labeled probes hybridized to the extended capture probes.

In some embodiments, steps (a)-(i) are performed under a first set of conditions, and the method includes repeating steps (a)-(i) under a second set of conditions.

Also provided herein are kits including (a) an array with a plurality of capture probes, where a capture probe of the plurality of capture probes includes a capture domain that specifically binds to an analyte in a biological sample; (b) a template switching oligonucleotide including a strand of nucleic acid including in a 5′ to 3′ direction: a primer binding sequence and a homopolymeric sequence; and (c) one or more labeled probes including the template switching oligonucleotide or a portion thereof.

In some kits, the capture probe of the plurality of capture probes includes a spatial barcode. In some kits, the capture probe of the plurality of capture probes includes one or more functional domains, a unique molecular identifier, a cleavage domain, or any combination thereof.

In some kits, the kit includes terminal deoxynucleotidyl transferase (TdT).

In some kits, the homopolymeric sequence is a poly(G) sequence.

In some kits, the kit includes one or both of a reverse transcriptase and a DNA polymerase.

In some kits, a labeled probe of the one or more labeled probes includes a fluorescent label.

In some kits, the kit includes one or more permeabilization reagents. In some kits, the kit includes one or more RNase inhibitors. In some kits, the kit includes one or more of: a protease, an RNase, a DNase, and a lipase.

In some kits, the kit includes one or more stains. In some kits, the one or more stains includes hematoxylin and eosin.

In some kits, the kit includes instructions for performing any one of the methods described herein.

All publications, patents, and patent applications mentioned in this specification are herein incorporated by reference to the same extent as if each individual publication, patent, patent application, or item of information was specifically and individually indicated to be incorporated by reference. To the extent publications, patents, patent applications, and items of information incorporated by reference contradict the disclosure contained in the specification, the specification is intended to supersede and/or take precedence over any such contradictory material.

Where values are described in terms of ranges, it should be understood that the description includes the disclosure of all possible sub-ranges within such ranges, as well as specific numerical values that fall within such ranges irrespective of whether a specific numerical value or specific sub-range is expressly stated.

The term “each,” when used in reference to a collection of items, is intended to identify an individual item in the collection but does not necessarily refer to every item in the collection, unless expressly stated otherwise, or unless the context of the usage clearly indicates otherwise.

Various embodiments of the features of this disclosure are described herein. However, it should be understood that such embodiments are provided merely by way of example, and numerous variations, changes, and substitutions can occur to those skilled in the art without departing from the scope of this disclosure. It should also be understood that various alternatives to the specific embodiments described herein are also within the scope of this disclosure.

### DESCRIPTION OF DRAWINGS

The following drawings illustrate certain embodiments of the features and advantages of this disclosure. These embodiments are not intended to limit the scope of the appended claims in any manner. Like reference symbols in the drawings indicate like elements.

FIGS. 1A-B is a schematic showing a captured analyte (e.g., mRNA) including crosslinks. The crosslinks can inhibit reverse transcription. FIG. 1A shows a captured analyte (e.g., mRNA) including crosslinks and in the absence of labelled nucleotides no signal is detected. Labelled nucleotides can be detected to indicate successful analyte capture (FIG. 1B).

FIG. 2 is a schematic showing a captured analyte (e.g., mRNA) without crosslinks. The captured analyte is reverse transcribed and a template switching oligonucleotide is incorporated into the extended probe. The incorporated template switching oligonucleotide is detected with a TSO-Cy3 conjugated probe.

FIGS. 3A-C show an exemplary scheme for determining the conditions in order to identify the most target analytes in a biological sample. FIG. 3A shows capture of an analyte, followed by reverse transcription, and a template switch oligonucleotide (TSO). FIG. 3B shows the cDNA strand after dehybridization and FIG. 3C shows a labeled probe hybridized to the TSO.

FIGS. 4A-B show replicate experiments using fixed-formalin paraffin-embedded (FFPE) mouse brain samples; hematoxylin and eosin (H&E) staining of the mouse brain samples (left), a background fluorescence scan after removal of the mouse brain samples (middle), and TSO-Cy3 conjugated fluorescent signal after hybridization (right).

FIGS. 5A-D show FFPE assay results in four soft tissue sarcoma tissue samples with H&E staining (left), a background fluorescence scan (middle), and a TSO-Cy3 signal (right) from; FIG. 5A) 37 year old FFPE sample, FIG. 5B) 18 year old FFPE sample, FIG. 5C) 10 year old FFPE sample, and FIG. 5D) 5 year old FFPE sample.

FIGS. 6A-B show replicate experimental results using an ovarian carcinosarcoma sample with H&E staining of the carcinosarcoma samples (left), a background fluorescence scan after removal of the carcinosarcoma samples (middle), and TSO-Cy3 signal after hybridization (right).

FIG. 7 shows an amplification plot for the soft tissue sarcoma samples identified in FIGS. 5A-D.

FIGS. 8A-D show cDNA amplification plots for each of the biological samples identified in FIGS. 5A-D.

FIGS. 9A-F show replicate experiments using FFPE colon cancer samples; H&E staining of the colon cancer samples (left), TO-Cy3 conjugated fluorescent signal after hybridization (middle), and decrosslinking conditions tested (right).

FIGS. 10A-C show TO-Cy3 conjugated fluorescent signal after hybridization in FFPE colon cancer samples under various decrosslinking conditions (top) followed by various permeabilization conditions (left).

FIGS. 11A-D show cDNA signal generated under various decrosslinking conditions followed by permeabilization for various times shown.

### DETAILED DESCRIPTION

Detecting analytes within a biological sample (e.g., fixed biological samples) includes determining optimal conditions for capturing analytes. Generally, captured analytes (e.g., captured with a capture probe as described herein) are reverse transcribed with labelled nucleotides and are detected under different conditions to determine the optimal conditions for performing spatial gene expression analysis. However, in some biological samples (e.g., fixed biological samples) analytes can be degraded and detection with labelled nucleotides during reverse transcription can be challenging. Thus, to remove the dependency of incorporating labelled nucleotides during cDNA synthesis the present disclosure features methods of detecting the template switch oligonucleotide (TSO) with one or more labelled probes complementary to the TSO.

For example, it is understood that template switching with a TSO primer occurs for all lengths of transcripts and not just full length analytes. In some biological samples, such as fixed biological samples, analytes can be degraded, truncated, and/or include crosslinks. To help identify optimal experimental conditions to capture degraded and/or truncated target analytes, methods to determine the efficiency of cDNA synthesis incorporation of the TSO sequence can be performed, for example by hybridizing and detecting one or more labeled probes to the TSO sequence. This method provides an advantage by determining the presence of truncated analytes in a biological sample, including within highly degraded biological samples (e.g., fixed biological samples). Generally, shorter sequences (about 50 to about 75 nucleotides) are sufficient to determine gene identity, thus the methods and kits disclosed herein provide a solution for determining the optimal conditions to detect analytes in a biological sample, including degraded analytes in a fixed biological sample.

Spatial analysis methodologies and compositions described herein can provide a vast amount of analyte and/or expression data for a variety of analytes within a biological sample at high spatial resolution, while retaining native spatial context. Spatial analysis methods and compositions can include, e.g., the use of a capture probe including a spatial barcode (e.g., a nucleic acid sequence that provides information as to the location or position of an analyte within a cell or a tissue sample (e.g., mammalian cell or a mammalian tissue sample) and a capture domain that is capable of binding to an analyte (e.g., a protein and/or a nucleic acid) produced by and/or present in a cell. Spatial analysis methods and compositions can also include the use of a capture probe having a capture domain that captures an intermediate agent for indirect detection of an analyte. For example, the intermediate agent can include a nucleic acid sequence (e.g., a barcode) associated with the intermediate agent. Detection of the intermediate agent is therefore indicative of the analyte in the cell or tissue sample.

Non-limiting aspects of spatial analysis methodologies and compositions are described in U.S. Pat. Nos. 10,774,374, 10,724,078, 10,480,022, 10,059,990, 10,041,949, 10,002,316, 9,879,313, 9,783,841, 9,727,810, 9,593,365, 8,951,726, 8,604,182, 7,709,198, U.S. Patent Application Publication Nos. 2020/239946, 2020/080136, 2020/0277663, 2020/024641, 2019/330617, 2019/264268, 2020/256867, 2020/224244, 2019/194709, 2019/161796, 2019/085383, 2019/055594, 2018/216161, 2018/051322, 2018/0245142, 2017/241911, 2017/089811, 2017/067096, 2017/029875, 2017/0016053, 2016/108458, 2015/000854, 2013/171621, WO 2018/091676, WO 2020/176788, Rodrigues et al., Science 363(6434):1463-1467, 2019; Lee et al., Nat. Protoc. 10(3):442-458, 2015; Trejo et al., PLoS ONE 14(2):e0212031, 2019; Chen et al., Science 348(6233):aaa6090, 2015; Gao et al., BMC Biol. 15:50, 2017; and Gupta et al., Nature Biotechnol. 36:1197-1202, 2018; the Visium Spatial Gene Expression Reagent Kits User Guide (e.g., Rev C, dated June 2020), and/or the Visium Spatial Tissue Optimization Reagent Kits User Guide (e.g., Rev C, dated July 2020), both of which are available at the 10× Genomics Support Documentation website, and can be used herein in any combination, and each of which is incorporated herein by reference in their entireties. Further non-limiting aspects of spatial analysis methodologies and compositions are described herein.

Some general terminology that may be used in this disclosure can be found in Section (I)(b) of WO 2020/176788 and/or U.S. Patent Application Publication No. 2020/0277663. Typically, a “barcode” is a label, or identifier, that conveys or is capable of conveying information (e.g., information about an analyte in a sample, a bead, and/or a capture probe). A barcode can be part of an analyte, or independent of an analyte. A barcode can be attached to an analyte. A particular barcode can be unique relative to other barcodes. For the purpose of this disclosure, an “analyte” can include any biological substance, structure, moiety, or component to be analyzed. The term “target” can similarly refer to an analyte of interest.

Analytes can be broadly classified into one of two groups: nucleic acid analytes, and non-nucleic acid analytes. Examples of non-nucleic acid analytes include, but are not limited to, lipids, carbohydrates, peptides, proteins, glycoproteins (N-linked or O-linked), lipoproteins, phosphoproteins, specific phosphorylated or acetylated variants of proteins, amidation variants of proteins, hydroxylation variants of proteins, methylation variants of proteins, ubiquitylation variants of proteins, sulfation variants of proteins, viral proteins (e.g., viral capsid, viral envelope, viral coat, viral accessory, viral glycoproteins, viral spike, etc.), extracellular and intracellular proteins, antibodies, and antigen binding fragments. In some embodiments, the analyte(s) can be localized to subcellular location(s), including, for example, organelles, e.g., mitochondria, Golgi apparatus, endoplasmic reticulum, chloroplasts, endocytic vesicles, exocytic vesicles, vacuoles, lysosomes, etc. In some embodiments, analyte(s) can be peptides or proteins, including without limitation antibodies and enzymes. Additional examples of analytes can be found in Section (I)(c) of WO 2020/176788 and/or U.S. Patent Application Publication No. 2020/0277663. In some embodiments, an analyte can be detected indirectly, such as through detection of an intermediate agent, for example, a ligation product or an analyte capture agent (e.g., an oligonucleotide-conjugated antibody), such as those described herein.

A “biological sample” is typically obtained from the subject for analysis using any of a variety of techniques including, but not limited to, biopsy, surgery, and laser capture microscopy (LCM), and generally includes cells and/or other biological material from the subject. In some embodiments, a biological sample can be a tissue section. In some embodiments, a biological sample can be a fixed and/or stained biological sample (e.g., a fixed and/or stained tissue section). Non-limiting examples of stains include histological stains (e.g., hematoxylin and/or eosin) and immunological stains (e.g., fluorescent stains). In some embodiments, a biological sample (e.g., a fixed and/or stained biological sample) can be imaged. Biological samples are also described in Section (I)(d) of WO 2020/176788 and/or U.S. Patent Application Publication No. 2020/0277663.

In some embodiments, a biological sample is permeabilized with one or more permeabilization reagents. For example, permeabilization of a biological sample can facilitate analyte capture. Exemplary permeabilization agents and conditions are described in Section (I)(d)(ii)(13) or the Exemplary Embodiments Section of WO 2020/176788 and/or U.S. Patent Application Publication No. 2020/0277663.

Array-based spatial analysis methods involve the transfer of one or more analytes from a biological sample to an array of features on a substrate, where each feature is associated with a unique spatial location on the array. Subsequent analysis of the transferred analytes includes determining the identity of the analytes and the spatial location of the analytes within the biological sample. The spatial location of an analyte within the biological sample is determined based on the feature to which the analyte is bound (e.g., directly or indirectly) on the array, and the feature's relative spatial location within the array.

A “capture probe” refers to any molecule capable of capturing (directly or indirectly) and/or labelling an analyte (e.g., an analyte of interest) in a biological sample. In some embodiments, the capture probe is a nucleic acid or a polypeptide. In some embodiments, the capture probe includes a barcode (e.g., a spatial barcode and/or a unique molecular identifier (UMI)) and a capture domain). In some embodiments, a capture probe can include a cleavage domain and/or a functional domain (e.g., a primer-binding site, such as for next-generation sequencing (NGS)). See, e.g., Section (II)(b) (e.g., subsections (i)-(vi)) of WO 2020/176788 and/or U.S. Patent Application Publication No. 2020/0277663. Generation of capture probes can be achieved by any appropriate method, including those described in Section (II)(d)(ii) of WO 2020/176788 and/or U.S. Patent Application Publication No. 2020/0277663.

In some embodiments, more than one analyte type (e.g., nucleic acids and proteins) from a biological sample can be detected (e.g., simultaneously or sequentially) using any appropriate multiplexing technique, such as those described in Section (IV) of WO 2020/176788 and/or U.S. Patent Application Publication No. 2020/0277663.

In some embodiments, detection of one or more analytes (e.g., protein analytes) can be performed using one or more analyte capture agents. As used herein, an “analyte capture agent” refers to an agent that interacts with an analyte (e.g., an analyte in a biological sample) and with a capture probe (e.g., a capture probe attached to a substrate or a feature) to identify the analyte. In some embodiments, the analyte capture agent includes: (i) an analyte binding moiety (e.g., that binds to an analyte), for example, an antibody or antigen-binding fragment thereof; (ii) analyte binding moiety barcode; and (iii) an analyte capture sequence. As used herein, the term “analyte binding moiety barcode” refers to a barcode that is associated with or otherwise identifies the analyte binding moiety. As used herein, the term “analyte capture sequence” refers to a region or moiety configured to hybridize to, bind to, couple to, or otherwise interact with a capture domain of a capture probe. In some cases, an analyte binding moiety barcode (or portion thereof) may be able to be removed (e.g., cleaved) from the analyte capture agent. Additional description of analyte capture agents can be found in Section (II)(b)(ix) of WO 2020/176788 and/or Section (II)(b)(viii) U.S. Patent Application Publication No. 2020/0277663.

There are at least two methods to associate a spatial barcode with one or more neighboring cells, such that the spatial barcode identifies the one or more cells, and/or contents of the one or more cells, as associated with a particular spatial location. One method is to promote analytes or analyte proxies (e.g., intermediate agents) out of a cell and towards a spatially-barcoded array (e.g., including spatially-barcoded capture probes). Another method is to cleave spatially-barcoded capture probes from an array and promote the spatially-barcoded capture probes towards and/or into or onto the biological sample.

In some cases, capture probes may be configured to prime, replicate, and consequently yield optionally barcoded extension products from a template (e.g., a DNA or RNA template, such as an analyte or an intermediate agent (e.g., a ligation product or an analyte capture agent), or a portion thereof), or derivatives thereof (see, e.g., Section (II)(b)(vii) of WO 2020/176788 and/or U.S. Patent Application Publication No. 2020/0277663 regarding extended capture probes). In some cases, capture probes may be configured to form ligation products with a template (e.g., a DNA or RNA template, such as an analyte or an intermediate agent, or portion thereof), thereby creating ligations products that serve as proxies for a template.

As used herein, an “extended capture probe” refers to a capture probe having additional nucleotides added to the terminus (e.g., 3′ or 5′ end) of the capture probe thereby extending the overall length of the capture probe. For example, an “extended 3′ end” indicates additional nucleotides were added to the most 3′ nucleotide of the capture probe to extend the length of the capture probe, for example, by polymerization reactions used to extend nucleic acid molecules including templated polymerization catalyzed by a polymerase (e.g., a DNA polymerase or a reverse transcriptase). In some embodiments, extending the capture probe includes adding to a 3′ end of a capture probe a nucleic acid sequence that is complementary to a nucleic acid sequence of an analyte or intermediate agent specifically bound to the capture domain of the capture probe. In some embodiments, the capture probe is extended using reverse transcription. In some embodiments, the capture probe is extended using one or more DNA polymerases. The extended capture probes include the sequence of the capture probe and the sequence of the spatial barcode of the capture probe.

In some embodiments, extended capture probes are amplified (e.g., in bulk solution or on the array) to yield quantities that are sufficient for downstream analysis, e.g., via DNA sequencing. In some embodiments, extended capture probes (e.g., DNA molecules) act as templates for an amplification reaction (e.g., a polymerase chain reaction).

Additional variants of spatial analysis methods, including in some embodiments, an imaging step, are described in Section (II)(a) of WO 2020/176788 and/or U.S. Patent Application Publication No. 2020/0277663. Analysis of captured analytes (and/or intermediate agents or portions thereof), for example, including sample removal, extension of capture probes, sequencing (e.g., of a cleaved extended capture probe and/or a cDNA molecule complementary to an extended capture probe), sequencing on the array (e.g., using, for example, in situ hybridization or in situ ligation approaches), temporal analysis, and/or proximity capture, is described in Section (II)(g) of WO 2020/176788 and/or U.S. Patent Application Publication No. 2020/0277663. Some quality control measures are described in Section (II)(h) of WO 2020/176788 and/or U.S. Patent Application Publication No. 2020/0277663.

Spatial information can provide information of biological and/or medical importance. For example, the methods and compositions described herein can allow for: identification of one or more biomarkers (e.g., diagnostic, prognostic, and/or for determination of efficacy of a treatment) of a disease or disorder; identification of a candidate drug target for treatment of a disease or disorder; identification (e.g., diagnosis) of a subject as having a disease or disorder; identification of stage and/or prognosis of a disease or disorder in a subject; identification of a subject as having an increased likelihood of developing a disease or disorder; monitoring of progression of a disease or disorder in a subject; determination of efficacy of a treatment of a disease or disorder in a subject; identification of a patient subpopulation for which a treatment is effective for a disease or disorder; modification of a treatment of a subject with a disease or disorder; selection of a subject for participation in a clinical trial; and/or selection of a treatment for a subject with a disease or disorder. Exemplary methods for identifying spatial information of biological and/or medical importance can be found in U.S. Patent Application Publication No. 2021/0140982A1, U.S. Patent Application No. 2021/0198741A1, and/or U.S. Patent Application No. 2021/0199660.

Spatial information can provide information of biological importance. For example, the methods and compositions described herein can allow for: identification of transcriptome and/or proteome expression profiles (e.g., in healthy and/or diseased tissue); identification of multiple analyte types in close proximity (e.g., nearest neighbor analysis); determination of up- and/or down-regulated genes and/or proteins in diseased tissue; characterization of tumor microenvironments; characterization of tumor immune responses; characterization of cells types and their co-localization in tissue; and identification of genetic variants within tissues (e.g., based on gene and/or protein expression profiles associated with specific disease or disorder biomarkers).

Typically, for spatial array-based methods, a substrate functions as a support for direct or indirect attachment of capture probes to features of the array. A “feature” is an entity that acts as a support or repository for various molecular entities used in spatial analysis. In some embodiments, some or all of the features in an array are functionalized for analyte capture. Exemplary substrates are described in Section (II)(c) of WO 2020/176788 and/or U.S. Patent Application Publication No. 2020/0277663. Exemplary features and geometric attributes of an array can be found in Sections (II)(d)(i), (II)(d)(iii), and (II)(d)(iv) of WO 2020/176788 and/or U.S. Patent Application Publication No. 2020/0277663.

Generally, analytes and/or intermediate agents (or portions thereof) can be captured when contacting a biological sample with a substrate including capture probes (e.g., a substrate with capture probes embedded, spotted, printed, fabricated on the substrate, or a substrate with features (e.g., beads, wells) comprising capture probes). As used herein, “contact,” “contacted,” and/or “contacting,” a biological sample with a substrate refers to any contact (e.g., direct or indirect) such that capture probes can interact (e.g., bind covalently or non-covalently (e.g., hybridize)) with analytes from the biological sample. Capture can be achieved actively (e.g., using electrophoresis) or passively (e.g., using diffusion). Analyte capture is further described in Section (II)(e) of WO 2020/176788 and/or U.S. Patent Application Publication No. 2020/0277663.

In some cases, spatial analysis can be performed by attaching and/or introducing a molecule (e.g., a peptide, a lipid, or a nucleic acid molecule) having a barcode (e.g., a spatial barcode) to a biological sample (e.g., to a cell in a biological sample). In some embodiments, a plurality of molecules (e.g., a plurality of nucleic acid molecules) having a plurality of barcodes (e.g., a plurality of spatial barcodes) are introduced to a biological sample (e.g., to a plurality of cells in a biological sample) for use in spatial analysis. In some embodiments, after attaching and/or introducing a molecule having a barcode to a biological sample, the biological sample can be physically separated (e.g., dissociated) into single cells or cell groups for analysis. Some such methods of spatial analysis are described in Section (III) of WO 2020/176788 and/or U.S. Patent Application Publication No. 2020/0277663.

During analysis of spatial information, sequence information for a spatial barcode associated with an analyte is obtained, and the sequence information can be used to provide information about the spatial distribution of the analyte in the biological sample. Various methods can be used to obtain the spatial information. In some embodiments, specific capture probes and the analytes they capture are associated with specific locations in an array of features on a substrate. For example, specific spatial barcodes can be associated with specific array locations prior to array fabrication, and the sequences of the spatial barcodes can be stored (e.g., in a database) along with specific array location information, so that each spatial barcode uniquely maps to a particular array location.

Alternatively, specific spatial barcodes can be deposited at predetermined locations in an array of features during fabrication such that at each location, only one type of spatial barcode is present so that spatial barcodes are uniquely associated with a single feature of the array. Where necessary, the arrays can be decoded using any of the methods described herein so that spatial barcodes are uniquely associated with array feature locations, and this mapping can be stored as described above.

When sequence information is obtained for capture probes and/or analytes during analysis of spatial information, the locations of the capture probes and/or analytes can be determined by referring to the stored information that uniquely associates each spatial barcode with an array feature location. In this manner, specific capture probes and captured analytes are associated with specific locations in the array of features. Each array feature location represents a position relative to a coordinate reference point (e.g., an array location, a fiducial marker) for the array. Accordingly, each feature location has an “address” or location in the coordinate space of the array.

Some exemplary spatial analysis workflows are described in the Exemplary Embodiments section of WO 2020/176788 and/or U.S. Patent Application Publication No. 2020/0277663. See, for example, the Exemplary embodiment starting with “In some non-limiting examples of the workflows described herein, the sample can be immersed . . . ” of WO 2020/176788 and/or U.S. Patent Application Publication No. 2020/0277663. See also, e.g., the Visium Spatial Gene Expression Reagent Kits User Guide (e.g., Rev C, dated June 2020), and/or the Visium Spatial Tissue Optimization Reagent Kits User Guide (e.g., Rev C, dated July 2020).

In some embodiments, spatial analysis can be performed using dedicated hardware and/or software, such as any of the systems described in Sections (II)(e)(ii) and/or (V) of WO 2020/176788 and/or U.S. Patent Application Publication No. 2020/0277663, or any of one or more of the devices or methods described in Sections Control Slide for Imaging, Methods of Using Control Slides and Substrates for, Systems of Using Control Slides and Substrates for Imaging, and/or Sample and Array Alignment Devices and Methods, Informational labels of WO 2020/123320.

Suitable systems for performing spatial analysis can include components such as a chamber (e.g., a flow cell or sealable, fluid-tight chamber) for containing a biological sample. The biological sample can be mounted for example, in a biological sample holder. One or more fluid chambers can be connected to the chamber and/or the sample holder via fluid conduits, and fluids can be delivered into the chamber and/or sample holder via fluidic pumps, vacuum sources, or other devices coupled to the fluid conduits that create a pressure gradient to drive fluid flow. One or more valves can also be connected to fluid conduits to regulate the flow of reagents from reservoirs to the chamber and/or sample holder.

The systems can optionally include a control unit that includes one or more electronic processors, an input interface, an output interface (such as a display), and a storage unit (e.g., a solid state storage medium such as, but not limited to, a magnetic, optical, or other solid state, persistent, writeable and/or re-writeable storage medium). The control unit can optionally be connected to one or more remote devices via a network. The control unit (and components thereof) can generally perform any of the steps and functions described herein. Where the system is connected to a remote device, the remote device (or devices) can perform any of the steps or features described herein. The systems can optionally include one or more detectors (e.g., CCD, CMOS) used to capture images. The systems can also optionally include one or more light sources (e.g., LED-based, diode-based, lasers) for illuminating a sample, a substrate with features, analytes from a biological sample captured on a substrate, and various control and calibration media.

The systems can optionally include software instructions encoded and/or implemented in one or more of tangible storage media and hardware components such as application specific integrated circuits. The software instructions, when executed by a control unit (and in particular, an electronic processor) or an integrated circuit, can cause the control unit, integrated circuit, or other component executing the software instructions to perform any of the method steps or functions described herein.

In some cases, the systems described herein can detect (e.g., register an image) the biological sample on the array. Exemplary methods to detect the biological sample on an array are described in WO 2021/102003 and/or U.S. patent application Ser. No. 16/951,854, each of which is incorporated herein by reference in their entireties.

Prior to transferring analytes from the biological sample to the array of features on the substrate, the biological sample can be aligned with the array. Alignment of a biological sample and an array of features including capture probes can facilitate spatial analysis, which can be used to detect differences in analyte presence and/or level within different positions in the biological sample, for example, to generate a three-dimensional map of the analyte presence and/or level. Exemplary methods to generate a two- and/or three-dimensional map of the analyte presence and/or level are described in PCT Application No. 2020/053655 and spatial analysis methods are generally described in WO 2021/102039 and/or U.S. patent application Ser. No. 16/951,864, each of which is incorporated herein by reference in their entireties.

In some cases, a map of analyte presence and/or level can be aligned to an image of a biological sample using one or more fiducial markers, e.g., objects placed in the field of view of an imaging system which appear in the image produced, as described in the Substrate Attributes Section, Control Slide for Imaging Section of WO 2020/123320, WO 2021/102005, and/or U.S. patent application Ser. No. 16/951,843, each of which is incorporated herein by reference in their entireties. Fiducial markers can be used as a point of reference or measurement scale for alignment (e.g., to align a sample and an array, to align two substrates, to determine a location of a sample or array on a substrate relative to a fiducial marker) and/or for quantitative measurements of sizes and/or distances.

Methods and Kits for Analyte Capture Determination with Labelled Oligonucleotide Probes

Determining the optimal conditions for detecting analytes within a biological sample (e.g., fixed biological samples) includes determining optimal conditions for capturing analytes. Generally, captured analytes (e.g., RNA) are reverse transcribed with labelled nucleotides and are detected under different conditions (e.g., a first set of experimental conditions, a second set of experimental conditions, etc.), in order to determine the optimal conditions for performing spatial analysis. However, in some biological samples (e.g., fixed biological samples) analytes can be degraded and detection with labelled nucleotides can be challenging due to degraded analytes and/or truncated length. In addition, analytes (e.g., mRNA) in fixed biological samples can include crosslinks from the fixation process. Generally, such crosslinks can inhibit or prevent an enzyme, such as a reverse transcriptase, from reverse transcribing the full-length of the captured analyte, or a portion thereof. Thus, as an alternative to incorporating labelled nucleotides during cDNA synthesis the present disclosure features methods of detecting the template switch oligonucleotide (TSO) itself incorporated into the extended probe with one or more labelled probes complementary to the TSO.

Another limitation of assays determining the optimal condition for analyte capture is the removal of the biological sample. Portions of the biological sample left on the substrate (e.g., slide) can give rise to autofluorescence and since reverse transcription (e.g., reverse transcription of captured analytes to generate extended probes) occurs prior to removal of the biological sample there is no opportunity to scan the substrate for an autofluorescence biological sample background image.

Formalin-fixation and paraffin-embedding (FFPE) of clinical specimens has been the preferred method for biological sample preservation for decades. FFPE is less expensive and easier to use than freezing-based methods, and also offers a high degree of preservation of morphological detail.

It is understood that template switching with a TSO primer occurs for all lengths of transcripts and not just full length analytes. In some biological samples, such as fixed biological samples, analytes can be degraded, truncated, and/or include crosslinks (e.g., crosslinks from fixation). Thus, the present disclosure features methods that include hybridizing one or more labeled probes to the TSO sequence incorporated into the extended probe (e.g., during reverse transcription). This method provides an advantage to determine the presence of truncated analytes in a biological sample, including within highly degraded biological samples (e.g., fixed biological samples, fixed biological samples containing analytes with crosslinks). Generally, shorter sequences (e.g., about 50 to about 75 nucleotides) are sufficient to determine gene identity, thus the methods and kits disclosed herein provide a solution for determining the optimal conditions to detect analytes in a degraded biological sample.

Provided herein are methods including (a) a biological sample on an array including a plurality of capture probes, where a capture probe includes a capture domain that specifically binds to an analyte in the biological sample; (b) extending a 3′ end of the capture probes using the analyte as a template to generate an extended capture probe; (c) adding to the 3′ end of the extended capture probes a first homopolymeric sequence; (d) contacting the extended capture probes with a template switching oligonucleotide including a strand of nucleic acid including in a 5′ to 3′ direction: a primer binding sequence and a second homopolymeric sequence, where the second homopolymeric sequence binds specifically to the first homopolymeric sequence; (e) extending the 3′ end of the extended capture probes using the template switching oligonucleotide, or a portion thereof, as a template; (0 removing the analyte and the template-switching oligonucleotides from the extended capture probes; (g) hybridizing one or more labeled probes including the template switching oligonucleotide or a portion thereof to the extended capture probes; and (h) detecting the labeled probes hybridized to extended capture probes.

In some embodiments, steps (a)-(h) are performed under a first set of conditions, and the method further includes repeating steps (a)-(h) under a second set of conditions.

Also provided herein are methods including (a) contacting a biological sample with an array including a plurality of capture probes, where a capture probe includes a capture domain that specifically binds to an analyte in the biological sample; (b) staining and imaging the biological sample; (c) extending a 3′ end of the capture probes using the analytes as a template to generate extended capture probes; (d) adding to the 3′ end of the extended capture probes a first homopolymeric sequence; (e) contacting the extended capture probes with a template switching oligonucleotide comprising in a 5′ to 3′ direction: a primer binding sequence and a second homopolymeric sequence, wherein the second homopolymeric sequence binds specifically to the first homopolymeric sequence; (0 extending the 3′ end of the extended capture probes using the template switching oligonucleotide as a template; (g) removing the analytes and the template-switching oligonucleotides from the extended capture probes; (h) hybridizing one or more labeled probes comprising the template switching oligonucleotide or a portion thereof to the extended capture probes; and (i) detecting the one or more labeled probes hybridized to the extended capture probes.

In some embodiments, steps (a)-(i) are performed under a first set of conditions, and the methods further include repeating steps (a)-(i) under a second set of conditions.

As described herein spatial analysis methods and compositions can include, e.g., the use of a capture probe including a capture domain that is capable of binding to an analyte (e.g., a protein or a nucleic acid) produced by and/or present in the biological sample.

Also described herein are spatial analysis methods and compositions that can include, e.g., the use of a capture probe including a spatial barcode (e.g., a nucleic acid sequence that provides information as to the location or position of an analyte within a cell or a tissue sample (e.g., mammalian cell or a mammalian tissue sample)) and a capture domain that is capable of binding to an analyte (e.g., a protein or a nucleic acid) produced by and/or present in the biological sample. In some embodiments, the capture domain includes a poly(T) sequence.

### Template Switching and Template Switching Oligonucleotides

A “template switching oligonucleotide” is an oligonucleotide that hybridizes to untemplated nucleotides added by a reverse transcriptase (e.g., enzyme with terminal transferase activity) during reverse transcription. In some embodiments, a template switching oligonucleotide hybridizes to untemplated poly(C) nucleotides added by a reverse transcriptase. In some embodiments, a template switching oligonucleotide hybridizes to untemplated poly(C) nucleotides added by terminal deoxynucleotidyl transferase (TdT). In some embodiments, the template switching oligonucleotide adds a common sequence to full-length cDNA that is used for cDNA amplification.

In some embodiments, the template switching oligonucleotide adds a common sequence onto the 5′ end of the RNA being reverse transcribed. For example, a template switching oligonucleotide can hybridize to untemplated poly(C) nucleotides added onto the end of a cDNA molecule (e.g., added by any of the methods described herein) and provide a template for the reverse transcriptase to continue replication to the 5′ end of the template switching oligonucleotide, thereby generating full-length cDNA ready for further amplification and/or downstream applications (e.g., sequencing). In some embodiments, once a full-length cDNA molecule is generated, the template switching oligonucleotide can serve as a primer in a cDNA amplification reaction.

For example, a first homopolymeric sequence can be added to an extended capture probe by either a reverse transcriptase or TdT. In some embodiments, the first homopolymeric sequence is a poly(C) sequence, however, it is appreciated that other homopolymeric sequences can be added to the extended capture probe. In some embodiments, the template switching oligonucleotide includes in a 5′ to 3′ direction a primer binding sequence and a second homopolymeric sequence. In some embodiments, the second homopolymeric sequence can bind to the first homopolymeric sequence. In some embodiments, the second homopolymeric sequence is a poly(G) sequence (e.g., the poly(G) sequence hybridizes to the added poly(C) sequence).

In some embodiments, a template switching oligonucleotide is added before, contemporaneously with, or after a reverse transcription, or other terminal transferase-based reaction. In some embodiments, a template switching oligonucleotide is incorporated in the extended probe during reverse transcription (e.g., included in the extended probe after analyte capture). In certain embodiments, methods of sample analysis using template switching oligonucleotides can involve the generation of nucleic acid products (e.g., extended probes, or complements thereof) from analytes of the biological sample, followed by further processing of the nucleic acid products with the template switching oligonucleotide.

Template switching oligonucleotides can include a hybridization region (e.g., a homopolymeric sequence) and a template region. The hybridization region can include any sequence capable of hybridizing to the target. In some embodiments, the hybridization region can, e.g., include a series of G bases to complement the overhanging C bases at the 3′ end of a cDNA molecule. The series of G bases can include 1 G base, 2 G bases, 3 G bases, 4 G bases, 5 G bases, or more than 5 G bases. The template sequence (e.g., primer binding sequence) can include any sequence to be incorporated into the cDNA. In other embodiments, the hybridization region can include at least one base in addition to at least one G base. In other embodiments, the hybridization can include bases that are not a G base. In some embodiments, the template region (e.g., primer binding sequence) includes at least 1 (e.g., at least 2, 3, 4, 5 or more) functional sequences. In some embodiments, the template region and hybridization region are separated by a spacer (e.g., a nucleotide sequence spacer).

Template switching oligonucleotides can include deoxyribonucleic acids; ribonucleic acids; modified nucleic acids including 2-aminopurine, 2,6-diaminopurine (2-amino-dA), inverted dT, 5-methyl dC, 2′-deoxylnosine, Super T (5-hydroxybutyn1-2′-deoxyuridine), Super G (8-aza-7-deazaguanosine), locked nucleic acids (LNAs), unlocked nucleic acids (UNAs, e.g., UNA-A, UNA-U, UNA-C, UNA-G), Iso-dG, Iso-dC, 2′ fluoro bases (e.g., Fluoro C, Fluoro U, Fluoro A, and Fluoro G), or any combination of the foregoing. In some embodiments, the length of a template switching oligonucleotide can be at least about 1, 2, 10, 20, 50, 75, 100, 150, 200, or 250 nucleotides or longer. In some embodiments, the length of a template switching oligonucleotide can be at most about 2, 10, 20, 50, 100, 150, 200, or 250 nucleotides or longer.

### Detectable Label

The terms “detectable label,” “optical label,” and “label” are used interchangeably herein to refer to a directly or indirectly detectable moiety that is associated with (e.g., conjugated to) a molecule to be detected (e.g., a capture probe, analyte, and/or TSO). The detectable label can be directly detectable by itself (e.g., radioisotope labels or fluorescent labels) or, in the case of an enzymatic label, can be indirectly detectable, e.g., by catalyzing chemical alterations of a chemical substrate compound or composition, which chemical substrate compound or composition is directly detectable. Detectable labels can be suitable for small scale detection and/or suitable for high-throughput screening. As such, suitable detectable labels include, but are not limited to, radioisotopes, fluorophores, chemiluminescent compounds, bioluminescent compounds, and dyes.

The detectable label can be qualitatively detected (e.g., optically or spectrally), or it can be quantified. Qualitative detection generally includes a detection method in which the existence or presence of the detectable label is confirmed, whereas quantifiable detection generally includes a detection method having a quantifiable (e.g., numerically reportable) value such as an intensity, duration, polarization, and/or other properties. In some embodiments, the detectable label is bound to a feature or to a capture probe associated with a feature. For example, detectably labeled features can include a fluorescent, a colorimetric, or a chemiluminescent label attached to a bead (see, for example, Rajeswari et al., J. Microbiol Methods 139:22-28, 2017, and Forcucci et al., J. Biomed Opt. 10:105010, 2015, the entire contents of each of which are incorporated herein by reference).

For example, detectable labels can be incorporated during nucleic acid polymerization or amplification (e.g., Cy5®-labelled nucleotides, such as Cy5®-dCTP). Any suitable detectable label can be used. In some embodiments, the detectable label is a fluorophore. For example, the fluorophore can be selected from a group that includes: 7-AAD (7-Aminoactinomycin D), Acridine Orange (+DNA), Acridine Orange (+RNA), Alexa Fluor® 350, Alexa Fluor® 430, Alexa Fluor® 488, Alexa Fluor® 532, Alexa Fluor® 546, Alexa Fluor® 555, Alexa Fluor® 568, Alexa Fluor® 594, Alexa Fluor® 633, Alexa Fluor® 647, Alexa Fluor® 660, Alexa Fluor® 680, Alexa Fluor® 700, Alexa Fluor® 750, Allophycocyanin (APC), AMCA/AMCA-X, 7-Aminoactinomycin D (7-AAD), 7-Amino-4-methylcoumarin, 6-Aminoquinoline, Aniline Blue, ANS, APC-Cy7, ATTO-TAG™ CBQCA, ATTO-TAG™ FQ, Auramine 0-Feulgen, BCECF (high pH), BFP (Blue Fluorescent Protein), BFP/GFP FRET, BOBO™-1/BO-PRO™-1, BOBO™-3/BO-PRO™-3, BODIPY® FL, BODIPY® TMR, BODIPY® TR-X, BODIPY® 530/550, BODIPY® 558/568, BODIPY® 564/570, BODIPY® 581/591, BODIPY® 630/650-X, BODIPY® 650-665-X, BTC, Calcein, Calcein Blue, Calcium Crimson™, Calcium Green-1™, Calcium Orange™, Calcofluor® White, 5-Carboxyfluoroscein (5-FAM), 5-Carboxynaphthofluoroscein, 6-Carboxyrhodamine 6G, 5-Carboxytetramethylrhodamine (5-TAMRA), Carboxy-X-rhodamine (5-ROX), Cascade Blue®, Cascade Yellow™, CCF2 (GeneBLAzer™), CFP (Cyan Fluorescent Protein), CFP/YFP FRET, Chromomycin A3, Cl-NERF (low pH), CPM, 6-CR 6G, CTC Formazan, Cy20, Cy30, Cy3.50, Cy50, Cy5.50, Cy7®, Cychrome (PE-Cy5), Dansylamine, Dansyl cadaverine, Dansylchloride, DAPI, Dapoxyl, DCFH, DHR, DiA (4-Di-16-ASP), DiD (Di1C18(5)), DIDS, Dil (DilC18(3)), DiO (DiOC18(3)), DiR (Di1C18(7)), Di-4 ANEPPS, Di-8 ANEPPS, DM-NERF (4.5-6.5 pH), DsRed (Red Fluorescent Protein), EBFP, ECFP, EGFP, ELF®-97 alcohol, Eosin, Erythrosin, Ethidium bromide, Ethidium homodimer-1 (EthD-1), Europium (III) Chloride, 5-FAM (5-Carboxyfluorescein), Fast Blue, Fluorescein-dT phosphoramidite, FITC, Fluo-3, Fluo-4, FluorX R, Fluoro-Gold™ (high pH), Fluoro-Gold™ (low pH), Fluoro-Jade, FM® 1-43, Fura-2 (high calcium), Fura-2/BCECF, Fura Red™ (high calcium), Fura Red™/Fluo-3, GeneBLAzer™ (CCF2), GFP Red Shifted (rsGFP), GFP Wild Type, GFP/BFP FRET, GFP/DsRed FRET, Hoechst 33342 & 33258, 7-Hydroxy-4-methylcoumarin (pH 9), 1,5 IAEDANS, Indo-1 (high calcium), Indo-1 (low calcium), Indodicarbocyanine, Indotricarbocyanine, JC-1, 6-JOE, JOJO™-1/JO-PRO™-1, LDS 751 (+DNA), LDS 751 (+RNA), LOLO™-1/LO-PRO™-1, Lucifer Yellow, LysoSensor™ Blue (pH 5), LysoSensor™ Green (pH 5), LysoSensor™ Yellow/Blue (pH 4.2), LysoTrackert Green, LysoTracker® Red, LysoTracker® Yellow, Mag-Fura-2, Mag-Indo-1, Magnesium Green™ Marina Blue®, 4-Methylumbelliferone, Mithramycin, MitoTrackert Green, MitoTrackert Orange, MitoTrackert Red, NBD (amine), Nile Red, Oregon Green® 488, Oregon Green® 500, Oregon Green® 514, Pacific Blue, PBF1, PE (R-phycoerythrin), PE-Cy5, PE-Cy7, PE-Texas Red, PerCP (Peridinin chlorophyll protein), PerCP-Cy5.5 (TruRed), PharRed (APC-Cy7), C-phycocyanin, R-phycocyanin, R-phycoerythrin (PE), PI (Propidium Iodide), PKH26, PKH67, POPO™-1/PO-PRO™-1, POPO™-3/PO-PRO™-3, Propidium Iodide (PI), PyMPO, Pyrene, Pyronin Y, Quantam Red (PE-Cy5), Quinacrine Mustard, R670 (PE-Cy5), Red 613 (PE-Texas Red), Red Fluorescent Protein (DsRed), Resorufin, RH 414, Rhod-2, Rhodamine B, Rhodamine Green™, Rhodamine Red™, Rhodamine Phalloidin, Rhodamine 110, Rhodamine 123, 5-ROX (carboxy-X-rhodamine), S65A, S65C, S65L, S65T, SBFI, SITS, SNAFL®-1 (high pH), SNAFL®-2, SNARF®-1 (high pH), SNARF®-1 (low pH), Sodium Green™, SpectrumAqua®, SpectrumGreen® #1, SpectrumGreen® #2, SpectrumOrange®, SpectrumRed®, SYTO® 11, SYTO® 13, SYTO® 17, SYTO® 45, SYTOX® Blue, SYTOX® Green, SYTOX® Orange, 5-TAMRA (5-Carboxytetramethylrhodamine), Tetramethylrhodamine (TRITC), Texas Red®/Texas Red®-X, Texas Red®-X (NHS Ester), Thiadicarbocyanine, Thiazole Orange, TOTO®-1/TO-PRO®-1, TOTO®-3/TO-PRO®-3, TO-PRO®-5, Tri-color (PE-Cy5), TRITC (Tetramethylrhodamine), TruRed (PerCP-Cy5.5), WW 781, X-Rhodamine (XRITC), Y66F, Y66H, Y66 W, YFP (Yellow Fluorescent Protein), YOYO®-1/YO-PRO®-1, YOYO®-3/YO-PRO®-3, 6-FAM (Fluorescein), 6-FAM (NHS Ester), 6-FAM (Azide), HEX, TAMRA (NHS Ester), Yakima Yellow, MAX, TET, TEX615, ATTO 488, ATTO 532, ATTO 550, ATTO 565, ATTO Rho101, ATTO 590, ATTO 633, ATTO 647N, TYE 563, TYE 665, TYE 705, 5′ IRDye® 700, 5′ IRDye® 800, 5′ IRDye® 800CW (NHS Ester), WellRED D4 Dye, WellRED D3 Dye, WellRED D2 Dye, Lightcycler® 640 (NHS Ester), and/or Dy 750 (NHS Ester).

For example, one or more labeled probes (e.g., labelled oligonucleotide probes) including the template switching oligonucleotide sequence (e.g., including the primer binding sequence), or a portion thereof, can hybridize to the extended capture probe (e.g., extended with a TSO). In some embodiments, one or more labeled probes comprise SEQ ID NO: 1. In some embodiments, the one or more labeled probes are detected. In some embodiments, different experimental conditions, such as permeabilization conditions, enzyme concentration conditions, timing of different steps in a protocol, etc. are tested on biological samples. In some embodiments, 2, 3, 4, 5, 6, 7, 8 or more different experimental conditions are tested on biological samples.

In some embodiments, after extending the capture probe, the analyte and the TSO are removed from the extended capture probe. In some embodiments, the analyte and TSO are removed by denaturation. In some embodiments, denaturation includes applying heat to the array. In some embodiments, removing the analyte and the TSO includes one or more washing steps (e.g., 2, 3, 4, 5 or more washing steps). In some embodiments, the one or more washing steps includes the use of an acidic buffer. In some embodiments, the acidic buffer includes KOH.

### Decrosslinking

Decrosslinking can involve removing embedding material. For example, removing paraffin-embedding material (e.g., deparaffinization) from the biological sample (e.g., tissue section) can be performed by incubating the biological sample in an appropriate solvent (e.g., xylene), followed by a series of rinses (e.g., ethanol of varying concentrations), and rehydration in water. In some embodiments, the biological sample can be dried following deparaffinization. In some embodiments, after the step of drying the biological sample, the biological sample can be stained (e.g., H&E stain, any of the variety of stains described herein). In some embodiments, after staining the biological sample, the sample can be imaged.

After a fixed (e.g., FFPE fixed, PFA fixed) biological sample has undergone deparaffinization, the fixed (e.g., FFPE, PFA) biological sample can be further processed. For example, fixed (e.g., FFPE fixed, PFA fixed) biological samples can be treated to remove crosslinks (e.g., decrosslinking), such as formaldehyde-induced crosslinks. In some embodiments, decrosslinking the crosslinks (e.g., formaldehyde-induced crosslinks) in the fixed (e.g., FFPE fixed, PFA fixed) biological sample can include treating the biological sample with heat. In some embodiments, decrosslinking the formaldehyde-induced crosslinks can include performing a chemical reaction. In some embodiments, decrosslinking the formaldehyde-induced crosslinks, can include treating the sample with a permeabilization reagent. In some embodiments, decrosslinking the formaldehyde-induced crosslinks can include heat, a chemical reaction, and/or permeabilization reagents.

In some embodiments, decrosslinking crosslinks (e.g., formaldehyde-induced crosslinks) can be performed in the presence of a buffer. In some embodiments, the buffer is PBS buffer. In some embodiments, the buffer is Tris-EDTA (TE) buffer (e.g., TE buffer for FFPE biological samples). In some embodiments, the buffer is Tris-HCl buffer (e.g., Tris-HCl buffer for PFA fixed biological samples). In some embodiments, the buffer includes a salt. In some embodiments, the salt is NaCl, KCl, or any other salt described herein. In some embodiments, the buffer includes salt at about a concentration of about 50 mM, about 60 mM, about 70 mM, about 80 mM, about 90 mM, about 100 mM, about 110 mM, about 120 mM, about 130 mM, about 140 mM, about 150 mM, about 160 mM, about 170 mM, about 180 mM, about 190 mM, about 120 mM, about 130 mM, about 140 mM, about 150 mM, about 160 mM, about 170 mM, about 180 mM, about 190 mM, about 200 mM, about 210 mM, about 220 mM, about 230 mM, about 240 mM, about 250 mM, about 260 mM, about 270 mM, about 280 mM, about 290 mM, about 300 mM, about 310 mM, about 320 mM, about 330 mM, about 340 mM, about 350 mM, about 360 mM, about 370 mM, about 380 mM, about 390 mM, about 400 mM, about 410 mM, about 420 mM, about 430 mM, about 440 mM, about 450 mM, about 460 mM, about 470 mM, about 480 mM, about 490 mM, about 500 mM, about 510 mM, about 520 mM, about 530 mM, about 540 mM, about 550 mM, about 560 mM, about 570 mM, about 580 mM, about 590 mM, or about 600 mM.

In some embodiments, the buffer (e.g., TE buffer, Tris-HCl buffer) has a pH of about 7.0 to about 10.0, about 7.0 to about 9.9, about 7.0 to about 9.8, about 7.0 to about 9.7, about 7.0 to about 9.6, about 7.0 to about 9.5, about 7.0 to about 9.4, about 7.0 to about 9.3, about 7.0 to about 9.2, about 7.0 to about 9.1, about 7.0 to about 9.0, about 7.0 to about 8.9, about 7.0 to about 8.8, about 7.0 to about 8.7, about 7.0 to about 8.6, about 7.0 to about 8.5, about 7.0 to about 8.4, about 7.0 to about 8.3, about 7.0 to about 8.2, about 7.0 to about 8.1, about 7.0 to about 8.0, about 7.0 to about 7.9, about 7.0 to about 7.8, about 7.0 to about 7.7, about 7.0 to about 7.6, about 7.0 to about 7.5, about 7.0 to about 7.4, about 7.0 to about 7.3, about 7.0 to about 7.2, about 7.1 to about 10.0, about 7.2 to about 9.6, about 7.2 to about 9.5, about 7.2 to about 9.4, about 7.2 to about 9.3, about 7.2 to about 9.2, about 7.2 to about 9.1, about 7.2 to about 9.0, about 7.2 to about 8.9, about 7.2 to about 8.8, about 7.2 to about 8.7, about 7.2 to about 8.6, about 7.2 to about 8.5, about 7.2 to about 8.4, about 7.2 to about 8.3, about 7.2 to about 8.2, about 7.2 to about 8.1, about 7.2 to about 8.0, about 7.2 to about 7.9, about 7.2 to about 7.8, about 7.2 to about 7.7, about 7.2 to about 7.6, about 7.2 to about 7.5, about 7.2 to about 7.4, about 7.4 to about 10.0, about 7.4 to about 9.9, about 7.4 to about 9.8, about 7.4 to about 9.7, about 7.4 to about 9.6, about 7.4 to about 9.5, about 7.4 to about 9.4, about 7.4 to about 9.3, about 7.4 to about 9.2, about 7.4 to about 9.1, about 7.4 to about 9.0, about 7.4 to about 8.9, about 7.4 to about 8.8, about 7.4 to about 8.7, about 7.4 to about 8.6, about 7.4 to about 8.5, about 7.4 to about 8.4, about 7.4 to about 8.3, about 7.4 to about 8.2, about 7.4 to about 8.1, about 7.4 to about 8.0, about 7.4 to about 7.9, about 7.4 to about 7.8, about 7.4 to about 7.7, about 7.4 to about 7.6, about 7.6 to about 10.0, about 7.6 to about 9.9, about 7.6 to about 9.8, about 7.6 to about 9.7, about 7.6 to about 9.6, about 7.6 to about 9.5, about 7.6 to about 9.4, about 7.6 to about 9.3, about 7.6 to about 9.2, about 7.6 to about 9.1, about 7.6 to about 9.0, about 7.6 to about 8.9, about 7.6 to about 8.8, about 7.6 to about 8.7, about 7.6 to about 8.6, about 7.6 to about 8.5, about 7.6 to about 8.4, about 7.6 to about 8.3, about 7.6 to about 8.2, about 7.6 to about 8.1, about 7.6 to about 8.0, about 7.6 to about 7.9, about 7.6 to about 7.8, about 7.8 to about 10.0, about 7.8 to about 9.9, about 7.8 to about 9.8, about 7.8 to about 9.7, about 7.8 to about 9.6, about 7.8 to about 9.5, about 7.8 to about 9.4, about 7.8 to about 9.3, about 7.8 to about 9.2, about 7.8 to about 9.1, about 7.8 to about 9.0, about 7.8 to about 8.9, about 7.8 to about 8.8, about 7.8 to about 8.7, about 7.8 to about 8.6, about 7.8 to about 8.5, about 7.8 to about 8.4, about 7.8 to about 8.3, about 7.8 to about 8.2, about 7.8 to about 8.1, about 7.8 to about 8.0, about 7.9 to about 10.0, about 7.9 to about 9.9, about 7.9 to about 9.8, about 8.0 to about 10.0, about 8.0 to about 9.9, about 8.0 to about 9.8, about 8.0 to about 9.7, about 8.0 to about 9.6, about 8.0 to about 9.5, about 8.0 to about 9.4, about 8.0 to about 9.3, about 8.0 to about 9.2, about 8.0 to about 9.1, 8.0 to about 9.0, about 8.0 to about 8.9, about 8.0 to about 8.8, about 8.0 to about 8.7, about 8.0 to about 8.6, about 8.0 to about 8.5, about 8.0 to about 8.4, about 8.0 to about 8.3, about 8.0 to about 8.2, about 8.1 to about 10.0, about 8.2 to about 9.9, about 8.2 to about 9.8, about 8.2 to about 9.7, about 8.2 to about 9.6, about 8.2 to about 9.5, about 8.2 to about 9.4, about 8.2 to about 9.3, about 8.2 to about 9.2, about 8.2 to about 9.1, about 8.2 to about 9.0, about 8.2 to about 8.9, about 8.2 to about 8.8, about 8.2 to about 8.7, about 8.2 to about 8.6, about 8.2 to about 8.5, about 8.2 to about 8.4, about 8.4 to about 10.0, about 8.4 to about 9.9, about 8.4 to about 9.8, about 8.4 to about 9.7, about 8.4 to about 9.6, about 8.4 to about 9.5, about 8.4 to about 9.4, about 8.4 to about 9.3, about 8.4 to about 9.2, about 8.4 to about 9.1, about 8.4 to about 9.0, about 8.4 to about 8.9, about 8.4 to about 8.8, about 8.4 to about 8.7, about 8.4 to about 8.6, about 8.6 to about 10.0, about 8.6 to about 9.9, about 8.6 to about 9.8, about 8.6 to about 9.7, about 8.6 to about 9.6, about 8.6 to about 9.5, about 8.6 to about 9.4, about 8.6 to about 9.3, about 8.6 to about 9.2, about 8.6 to about 9.1, about 8.6 to about 9.0, about 8.6 to about 8.9, about 8.6 to about 8.8, about 8.8 to about 10.0, about 8.8 to about 9.9, about 8.8 to about 9.8, about 8.8 to about 9.7, about 8.8 to about 9.6, about 8.8 to about 9.5, about 8.8 to about 9.4, about 8.8 to about 9.3, about 8.8 to about 9.2, about 8.8 to about 9.1, about 8.8 to about 9.0, about 8.9 to about 10.0, about 8.9 to about 9.9, about 8.9 to about 9.8, about 8.9 to about 9.7, about 8.9 to about 9.6, about 8.9 to about 9.5, about 8.9 to about 9.4, about 8.9 to about 9.3, about 8.9 to about 9.2, or about 8.9 to about 9.1.

Permeabilization

In some embodiments, a biological sample can be permeabilized to facilitate transfer of analytes to the array. If a biological sample is not permeabilized sufficiently, the amount of analyte captured from the biological sample may be too low to enable adequate analysis. Conversely, if the biological sample is too permeable, the relative spatial relationship of the analytes within the biological sample can be lost. Hence, a balance between permeabilizing the biological sample enough to obtain good signal intensity while still maintaining the spatial resolution of the analyte distribution in the sample is desirable.

In general, a biological sample can be permeabilized by exposing the biological sample to one or more permeabilizing agents. Suitable agents for this purpose include, but are not limited to, organic solvents (e.g., acetone, ethanol, and methanol), cross-linking agents (e.g., paraformaldehyde), detergents (e.g., saponin, Triton X100™, Tween-20™, or sodium dodecyl sulfate (SDS)), and/or enzymes (e.g., trypsin, proteases (e.g., proteinase K)). In some embodiments, the detergent is an anionic detergent (e.g., SDS or N-lauroylsarcosine sodium salt solution). In some embodiments, the biological sample can be permeabilized using any of the methods described herein (e.g., using any of the detergents described herein, e.g., SDS and/or N-lauroylsarcosine sodium salt solution) before or after enzymatic treatment (e.g., treatment with any of the enzymes described herein, e.g., trypsin, proteases, collagenase (e.g., pepsin and/or proteinase K and/or collagenase)). In some embodiments, RecJ_{f} is included with the permeabilization agents.

In some embodiments, a biological sample can be permeabilized by exposing the sample to greater than about 1.0 w/v % (e.g., greater than about 2.0 w/v %, greater than about 3.0 w/v %, greater than about 4.0 w/v %, greater than about 5.0 w/v %, greater than about 6.0 w/v %, greater than about 7.0 w/v %, greater than about 8.0 w/v %, greater than about 9.0 w/v %, greater than about 10.0 w/v %, greater than about 11.0 w/v %, greater than about 12.0 w/v %, or greater than about 13.0 w/v %) sodium dodecyl sulfate (SDS) and/or N-lauroylsarcosine or N-lauroylsarcosine sodium salt. In some embodiments, a biological sample can be permeabilized by exposing the sample (e.g., for about 5 minutes to about 1 hour, about 5 minutes to about 50 minutes, about 5 minutes to about 40 minutes, about 5 minutes to about 30 minutes, about 5 minutes to about 20 minutes, or about 5 minutes to about 10 minutes) to about 1.0 w/v % to about 14.0 w/v % (e.g., about 2.0 w/v % to about 14.0 w/v %, about 2.0 w/v % to about 12.0 w/v %, about 2.0 w/v % to about 10.0 w/v %, about 4.0 w/v % to about 14.0 w/v %, about 4.0 w/v % to about 12.0 w/v %, about 4.0 w/v % to about 10.0 w/v %, about 6.0 w/v % to about 14.0 w/v %, about 6.0 w/v % to about 12.0 w/v %, about 6.0 w/v % to about 10.0 w/v %, about 8.0 w/v % to about 14.0 w/v %, about 8.0 w/v % to about 12.0 w/v %, about 8.0 w/v % to about 10.0 w/v %, about 10.0% w/v % to about 14.0 w/v %, about 10.0 w/v % to about 12.0 w/v %, or about 12.0 w/v % to about 14.0 w/v %) SDS and/or N-lauroylsarcosine salt solution and/or proteinase K (e.g., at a temperature of about 4% to about 35° C., about 4° C. to about 25° C., about 4° C. to about 20° C., about 4° C. to about 10° C., about 10° C. to about 25° C., about 10° C. to about 20° C., about 10° C. to about 15° C., about 35° C. to about 50° C., about 35° C. to about 45° C., about 35° C. to about 40° C., about 40° C. to about 50° C., about 40° C. to about 45° C., or about 45° C. to about 50° C.).

In some embodiments, the biological sample can be incubated with a permeabilizing agent to facilitate permeabilization of the sample. Additional methods for sample permeabilization are described, for example, in Jamur et al., Method Mol. Biol. 588:63-66, 2010, the entire contents of which are incorporated herein by reference.

In some embodiments, the biological sample is removed from the array. In some embodiments, the biological sample is removed from the array after permeabilization. In some embodiments, the biological sample is removed after step (b) as described herein.

In some embodiments, the biological sample is a tissue sample. In some embodiments, the tissue sample is a tissue section. In some embodiments, the tissue sample is a fixed tissue sample. In some embodiments, the fixed tissue sample is a fixed tissue section. In some embodiments, the fixed tissue sample is formalin-fixed paraffin-embedded (FFPE) tissue section. In some embodiments, the fixed tissue sample is a paraformaldehyde fixed tissue section. In some embodiments, the fixed tissue sample is an acetone fixed tissue section. In some embodiments, the fixed tissue sample is a methanol fixed tissue section.

In some embodiments, the biological sample is a mouse brain sample. In some embodiments, the biological sample is a soft tissue sarcoma sample. In some embodiments, the biological sample is an ovarian carcinosarcoma sample. In some embodiments, the biological sample is an organoid.

### Staining and Imaging

To facilitate visualization biological samples can be stained using a wide variety of stains and staining techniques. In some embodiments, a biological sample can be stained using any number of biological stains, including but not limited to, acridine orange, Bismarck brown, carmine, coomassie blue, cresyl violet, DAPI, eosin, ethidium bromide, acid fuchsine, hematoxylin, Hoechst stains, iodine, methyl green, methylene blue, neutral red, Nile blue, Nile red, osmium tetroxide, propidium iodide, rhodamine, and/or safranin.

The biological sample can be stained using known staining techniques, including Can-Grunwald, Giemsa, hematoxylin and eosin (H&E), Jenner's, Leishman, Masson's trichrome, Papanicolaou, Romanowsky, silver, Sudan, Wright's, and/or Periodic Acid Schiff (PAS) staining techniques. PAS staining is typically performed after formalin or acetone fixation.

Any variety of staining and imaging techniques as described herein or known in the art can be used in accordance with methods described herein. In some embodiments, the biological sample can be stained using a detectable label (e.g., radioisotopes, fluorophores, chemiluminescent compounds, bioluminescent compounds, and dyes) as described elsewhere herein. In some embodiments, a biological sample is stained using only one type of stain or one technique. In some embodiments, staining includes biological staining techniques such as H&E staining. In some embodiments, staining includes identifying analytes using fluorescently-conjugated antibodies. In some embodiments, a biological sample is stained using two or more different types of stains, or two or more different staining techniques. For example, a biological sample can be prepared by staining and imaging using one technique (e.g., H&E staining and brightfield imaging), followed by staining and imaging using another technique (e.g., IHC/IF staining and fluorescence microscopy) for the same biological sample.

In some embodiments, the one or more labeled probes are imaged by one or more of light field, bright field, dark field, phase contrast, fluorescence microscopy, reflection, interference, and/or confocal microscopy. In some embodiments, the one or more labeled probes are imaged by fluorescent microscopy.

### Kits

Also provided herein are kits including (a) an array with a plurality of capture probes, where a capture probe of the plurality of capture probes comprises a capture domain that specifically binds to an analyte in a biological sample; (b) a template switching oligonucleotide comprising a strand of nucleic acid comprising in a 5′ to 3′ direction: a primer binding sequence and a homopolymeric sequence; and (c) one or more labeled probes comprising the primer binding sequence or a portion thereof.

In some kits, the capture probe of the plurality of capture probes includes a spatial barcode. In some kits, the capture probe of the plurality of capture probes includes one or more functional domains, a unique molecular identifier, a cleavage domain, or any combination thereof.

In some kits, the kit includes a terminal deoxynucleotidyl transferase (TdT).

In some kits, the homopolymeric sequence is a poly(G) sequence.

In some kits, the kit includes a reverse transcriptase and/or a DNA polymerase.

In some kits, a labeled probe of the one or more labeled probes includes a fluorescent label.

In some kits, the kit includes one or more permeabilization reagents. In some kits, the kit includes one or more RNase inhibitors. In some kits, the kit includes one or more of: a protease, an RNase, a DNase, and a lipase.

In some kits, the kit includes one or more stains. In some kits, the one or more stains comprises hematoxylin and eosin.

In some kits, the kit includes instructions for performing any of the methods described herein.

### EXAMPLES

### Example 1. Labelled Oligonucleotide Probes for TSO Identification in Biological Samples

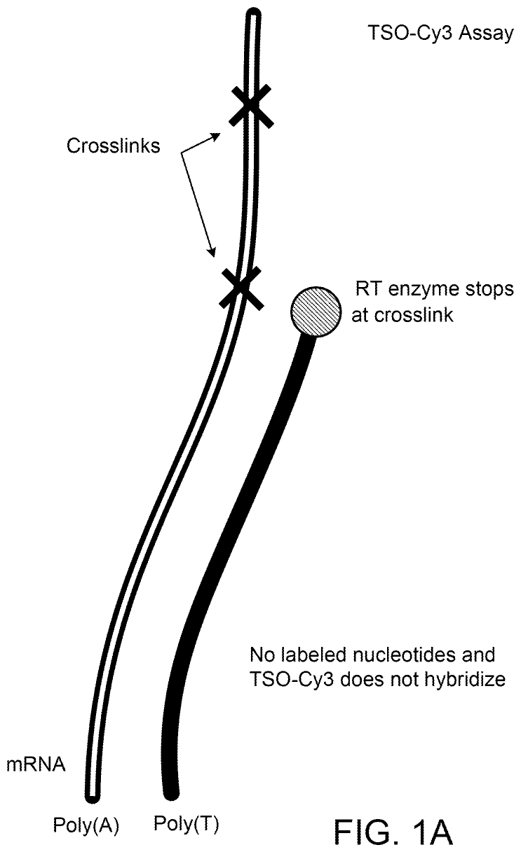

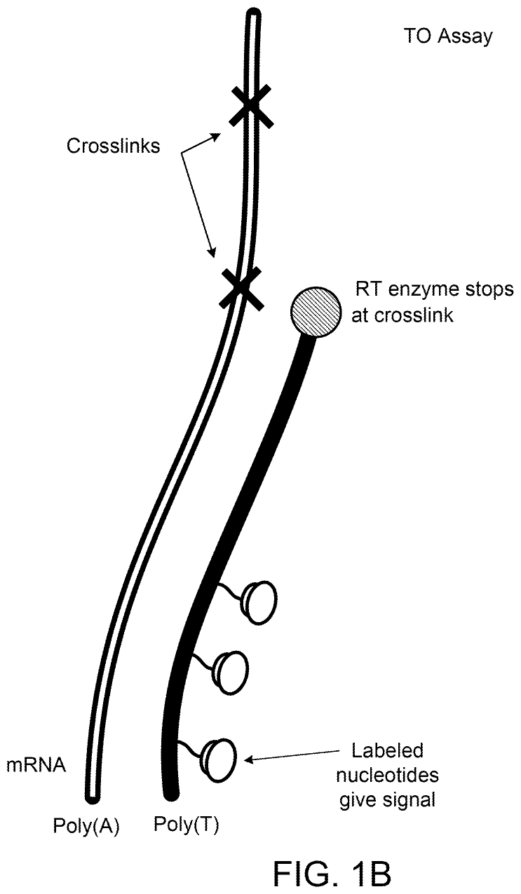

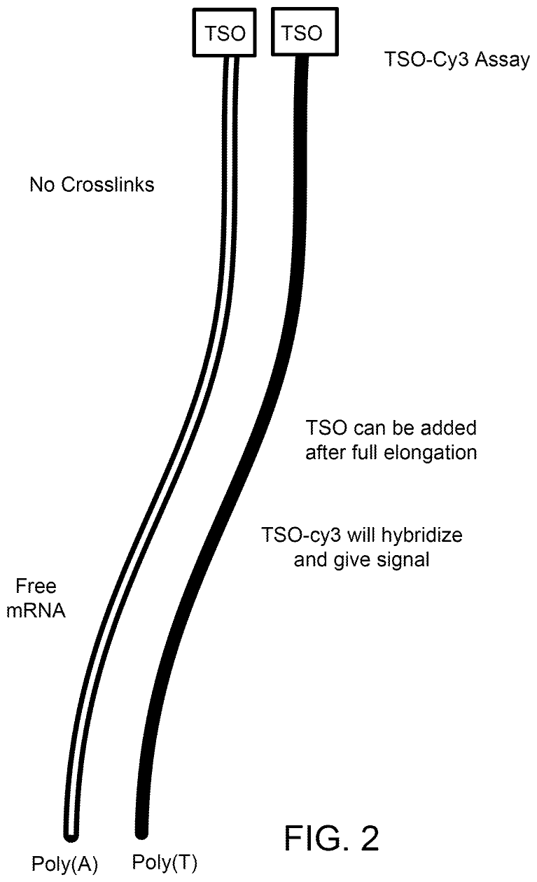

FIGS. 1A-B show the poly(T) portion of a capture probe hybridized to the poly(A) tail of an mRNA and both figures also show crosslinks present in the mRNA. FIG. 1A shows extension of the capture probe (e.g., generating an extended probe) by reverse transcription without labeled nucleotides. The reverse transcription reaction stops at the crosslinks resulting in a truncated extended probe that does not include the template switching oligonucleotide. FIG. 1B shows extension of the capture probe (e.g., generating an extended probe) by reverse transcription with labeled nucleotides. Incorporating labeled nucleotides into the extended probe is one way tissue optimization conditions (e.g., permeabilization) can be tested. However, incorporation of labeled nucleotides into the extended probe does not necessarily indicate that a template switching oligonucleotide has been incorporated into the extended probe. For example, the labeled nucleotides shown in FIG. 1B can be detected, however, the extended probe does not include the template switching oligonucleotide since the reverse transcriptase stops at the first encountered crosslink present in the mRNA. FIG. 2 shows a template switching oligonucleotide (TSO) Cy3 assay, where mRNA without crosslinks are captured by a capture probe and the capture probe is extended using the mRNA as a template. Since no crosslinks are present in the mRNA the reverse transcriptase extends the capture probe and a template switching oligonucleotide is incorporated into the extended probe at its 3′ end as described herein. The analyte (e.g., mRNA) can be removed and a labeled TSO oligonucleotide hybridizes to the TSO sequence present in the extended probe.

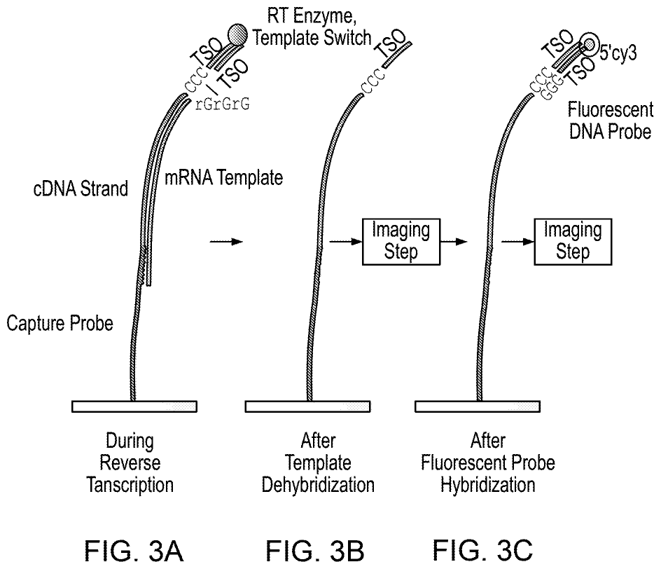

FIGS. 3A-C show an exemplary TSO labelling scheme in additional detail. FIG. 3A shows hybridization of an analyte (e.g., mRNA) with a capture probe and generation of first strand cDNA (e.g., an extended probe). At the 3′ of the cDNA untemplated nucleotides (d(C)) are added by a reverse transcriptase or a template independent polymerase such as TdT. A TSO is hybridized to the untemplated d(C) nucleotides where the TSO includes an adapter sequence, thus extending the capture probe (e.g., extending the first strand cDNA). FIG. 3B shows the extended probe (e.g., cDNA), after dehybridization, with a sequence complementary to the TSO incorporated into the extended probe. Following an imaging step, one or more fluorescently labeled probes (e.g., labeled with a Cy3 fluorophore) are contacted with the extended probe as shown in FIG. 3C and the fluorescently labeled extended probes are re-imaged. This assay ensures that the TSO has been incorporated into the extended probe(s), particularly in biological samples, such as fixed biological samples where the analytes (e.g., mRNA) can be degraded and/or include crosslinks which inhibit reverse transcription.

### Example 2. TSO Labelling Assay in Fixed Biological Samples

10 μm microtome sections of FFPE biological samples were prepared and incubated in a 40° C. water bath and attached to a slide. The FFPE biological samples were dried at 40° C. for 2 hours and placed in a refrigerator overnight. The FFPE biological samples were deparaffinized and rehydrated according to the following protocol: 2×15-minute washes in xylene; 2×2-minute washes in 100% ethanol, 2×2-minute wash in 96% ethanol, 2×2-minute wash in 70% ethanol, and 1×5-minute wash in H₂O. After deparaffinizing and rehydrating, the FFPE biological samples were H&E stained, dried, and imaged.

FFPE biological samples were pre-permeabilized with collagenase (0.2 U/μL) at 37° C. for 20 minutes prior to decrosslinking formaldehyde-induced crosslinks. To remove formaldehyde-induced crosslinks and/or modifications, FFPE biological samples were incubated in TE buffer (10 mM Tris, 1 mM EDTA, at pH 8.0) at 70° C. for 60 minutes. Following incubation in TE buffer, the FFPE biological samples were permeabilized with pepsin for 30 minutes. Analytes from the decrosslinked FFPE biological samples were captured by capture probes on the array substrate followed by reverse transcription (45 minutes at 53° C.) to extend the 3′ end of a capture probe using captured mRNA as a template.

After reverse transcription, the tissue was removed according to Step 2.0 in the Visium Spatial Tissue Optimization Reagent Kits User Guide (e.g., Rev D, dated Oct. 2020), followed by washes (2×SSC and 0.1% SDS at 50° C., followed by 0.2× SSC at room temperature, and 0.1×SSC at room temperature).

Background imaging was performed using an Innoscan 910 (Inopsys with a laser excitation of 532 nm, 5 μm per pixel resolution, and Gain 20 and 50 in two successive imaging steps).

Denaturation of the analyte and TSO was performed by a KOH wash step as described in Visium Spatial Gene Expression Reagent Kit User Guide CG000239 Rev C. The elution buffer was pipetted off and 75 μl of TSO-cy3 probe Mix was added: 162 μL MQ, 169 μL (20 mM Tris-HCl2 mM EDTA, 100 mM NaCl buffer), 6.75 μL 100 μM cy3-TSO probe (Integrated DNA Technologies, Sequence /5Cy3/AAGCAGTGGTATCAACGCAGAGTACATGGG (SEQ ID NO: 1), Purification HPLC). The slides were sealed and incubated in a thermocycler with the following program: step 1: 75° C. 1 s, step 2: 63° C. 15 s, step 3: 23° C. 15 min, step 4: 4° C. ∞, heated lid temperature: 80° C., reaction volume: 75 μl.

Signal imaging (e.g., Cy3 labeled probes) was performed with Cy3 labeled probes (SEQ ID NO: 1) prepared as shown in Table 1. Signal imaging was performed as described above for background fluorescence scanning.

TABLE 1

| | | |
|---|---|---|
| H20 | 43.25 | μl |
| Hybridization Buffer 2x | 50 | μl |
| (20 mM Tris-HCl, 2 mM | | |
| EDTA, 100 mM NaCl) | | |
| Cy3-TSO probe (100 | 6.75 | μl |
| μM) | | |

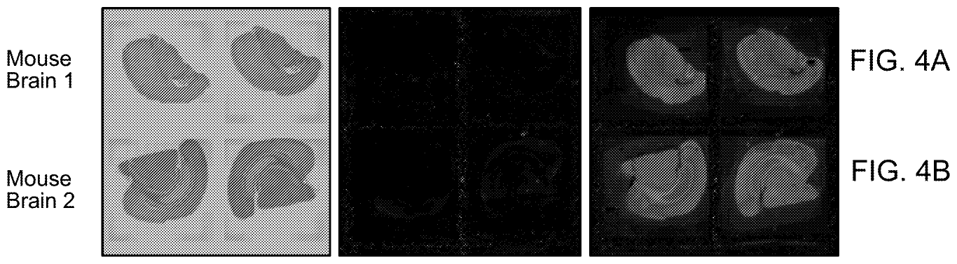

FIGS. 4A-B show replicate FFPE sample assays. H&E staining of mouse brain biological samples (left), a background fluorescence scan of the mouse brain biological samples (middle), and Cy3 labeled signal after hybridization of the TSO-Cy3 probes (right). The data show that a Cy3 labelled TSO probe effectively detects first strand cDNA (e.g., extended probes) where a TSO sequence was incorporated into the first strand cDNA from FFPE mouse brain samples, with little to no background fluorescence.

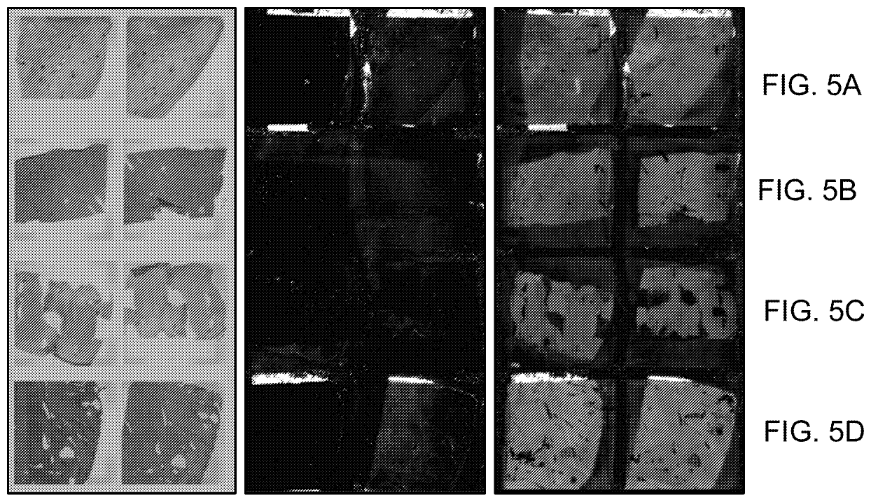

FIGS. 5A-D show FFPE sample assays in four soft tissue human sarcoma samples of different ages with H&E staining (left), background fluorescence scan (middle), and Cy3 labeled signal (right) for each soft tissue sarcoma sample. The signal imaging shown for TSO-Cy3 signal images (right) has a resolution of 5 μm/pixel with laser excitation of 532 nm. The soft tissue sarcoma samples were derived from a 37 year old FFPE sample (FIG. 5A), an 18 year old FFPE sample (FIG. 5B), a 10 year old FFPE sample (FIG. 5C), and a 5 year old FFPE sample (FIG. 5D). The data show that Cy3 labelled TSO probes effectively detect the addition of a TSO sequence to the first strand cDNA (e.g., extended probes) from captured mRNA from each of the biological samples similar to the H&E stained images, with little to no background fluorescence.

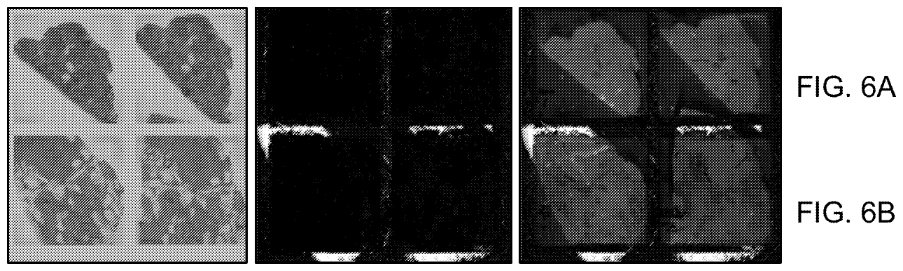

FIGS. 6A-B show replicate experiments of FFPE sample assays using ovarian carcinosarcoma samples. H&E staining of the carcinosarcoma samples (left), background fluorescence scans after removal of the carcinosarcoma samples (middle), and Cy3 fluorescent signal after hybridization (right). As with the mouse brain and soft tissue sarcoma samples, the method of TSO fluorescent labeling as a measure of the success of extending capture probes that has incorporated a TSO sequence when the analytes may be degraded, such as found in FFPE biological samples, was successful.

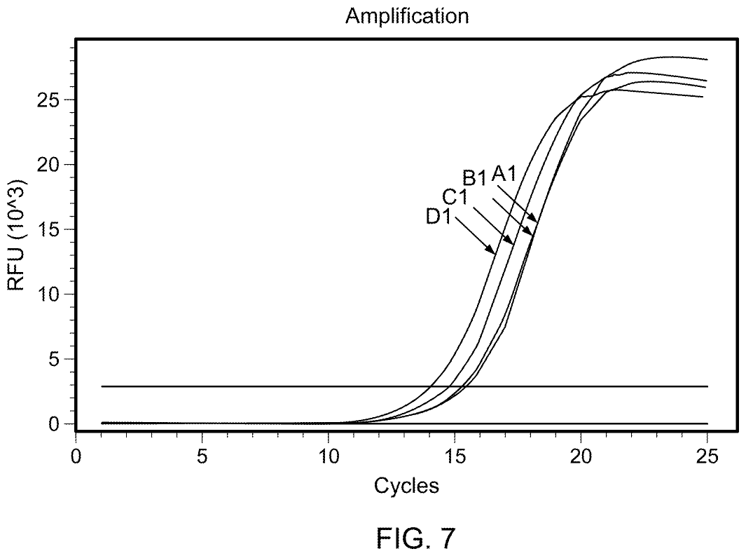

FIG. 7 shows a successful experimental amplification plot for each of the four soft tissue sarcoma samples described in FIGS. 5A-D. For example, A1 shown in FIG. 7 corresponds to the soft tissue sarcoma sample in FIG. 5A, B1 corresponds to the soft tissue sarcoma sample in FIG. 5B, Cl corresponds to the soft tissue sarcoma sample in FIG. 5C, and D1 corresponds to the soft tissue sarcoma sample in FIG. 5D. Results demonstrate that mRNA capture and extension was successful.

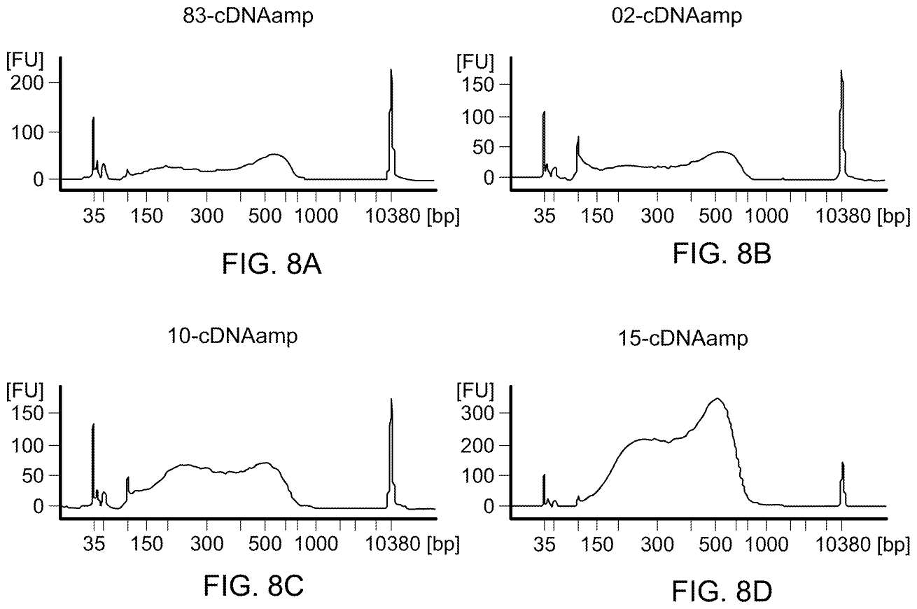

FIGS. 8A-D shows that cDNA was generated from each of the four soft tissue sarcoma samples described in FIGS. 5A-D. For example, FIG. 8A corresponds to the soft tissue sarcoma sample in FIG. 5A, FIG. 8B corresponds to the soft tissue sarcoma sample in FIG. 5B, FIG. 8C corresponds to the soft tissue sarcoma sample in FIG. 5C, and FIG. 8D corresponds to the soft tissue sarcoma sample in FIG. 5D. Results support those from FIG. 7 as to the success of capture, extension, and TSO sequence addition of mRNA from FFPE samples.

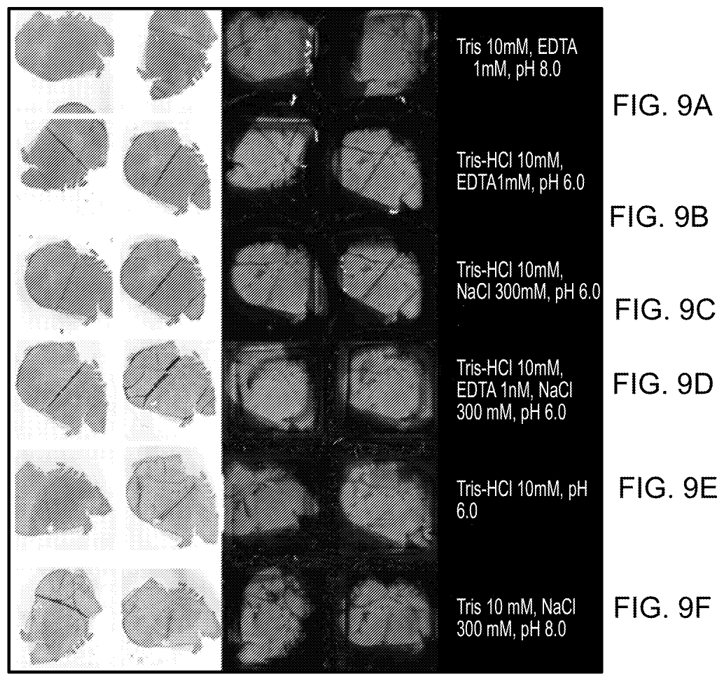

FIGS. 9A-F show H&E images of colon cancer tissue sections (left), cDNA signal observed after the tissue assay described above (center), and the decrosslinking buffer tested (right). Decrosslinking conditions were tested with various TE buffer (e.g., Tris and EDTA) compositions and pH using colon cancer FFPE tissues stored at 6° C. (FIGS. 9A-F). More specifically, TE or TE-HCl, pH, and NaCl concentrations were tested in duplicate. The following decrosslinking combinations were tested: Tris 10 mM, EDTA mM, pH 8.0 (FIG. 9A); Tris-HCl mM, EDTA 1 mM, pH 6.0 (FIG. 9B); Tris-HCl, 10 mM NaCl, 300 mM, pH 6.0 (FIG. 9C), Tris-HCl 10 mM, EDTA 1 mM, NaCl 300 mM, pH 6.0 (FIG. 9D); Tris-HCl 10 mM, pH 6.0 (FIG. 9E); Tris 10 mM, NaCl 300 mM, pH 8.0 (FIG. 9F). Each decrosslinking buffer was added to each well in duplicate and incubated at 70° C. for 1 hour.

The results demonstrate that the various combinations of pH, EDTA, and NaCl, all sufficiently decrosslinked the tissue sample as shown by the fluorescently-labelled cDNA signals performed during tissue optimization assays.

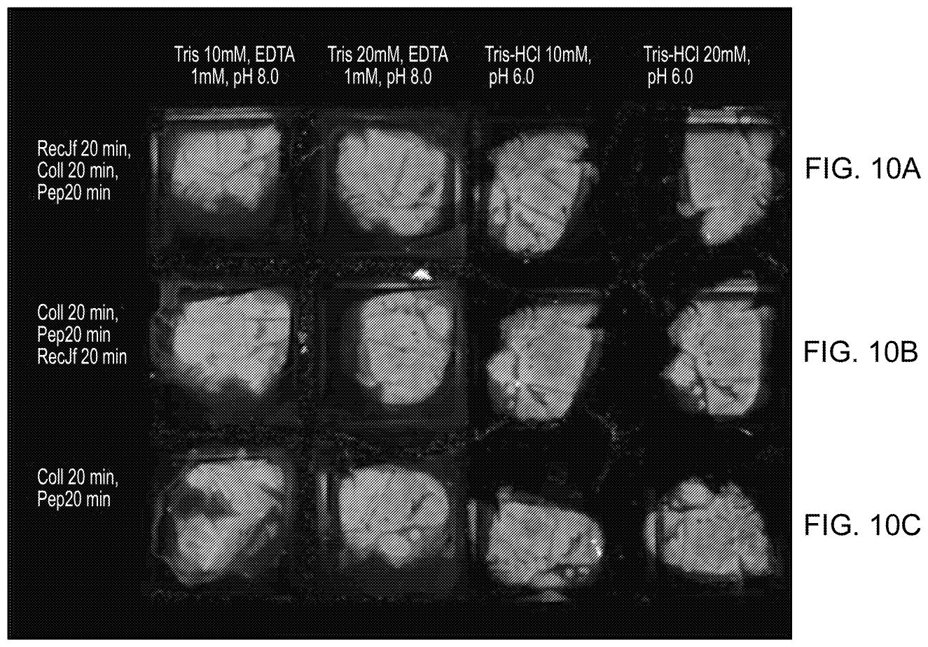

FIGS. 10A-C show cDNA generated under various decrosslinking conditions and various permeabilization conditions. More specifically, optimal decrosslinking conditions identified in FIGS. 9A-F, were tested further. The samples were decrosslinked with the buffers shown at the top of each column in FIGS. 10A-C (e.g., Tris 10 mM, EDTA 1 mM, pH 8.0; Tris 20 mM, EDTA 1 mM, pH 8.0; Tris-HCl 10 mM, pH 6.0; or Tris-HCl 20 mM, pH 6.0). Following decrosslinking the samples were permeabilized by a combination of collagenase, pepsin, and/or RecJ_{f}. Additionally, the order in which the permeabilization agents were added was also tested as follows: RecJ_{f} 20 minutes, collagenase 20 minutes, and pepsin 20 minutes (FIG. 10A); collagenase 20 minutes, pepsin 20 minutes, RecJ_{f} 20 minutes (FIG. 10B); and collagenase 20 minutes and pepsin 20 minutes (FIG. 10C).

The results demonstrate that the various combinations of decrosslinking buffers followed by various permeabilization agents, including the order in which the permeabilization agents were added to the sample, did not affect the ability to generate fluorescently-labelled cDNA signals.

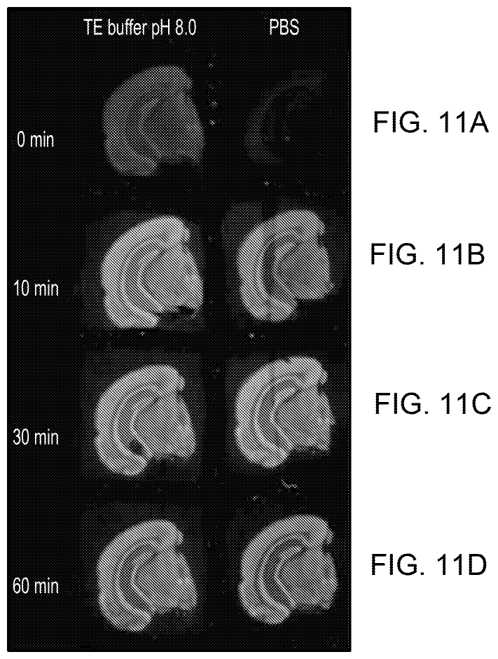

FIGS. 11A-D show cDNA signal generated under various decrosslinking conditions. More specifically, FIGS. 11A-D tested whether PBS can be used as a decrosslinking buffer by comparing PBS (right) with TE buffer at pH 8.0 (left). Following decrosslinking, mouse brain samples were permeabilized with pepsin for varying lengths of times: 0 minutes (FIG. 11A), 10 minutes (FIG. 11B), 30 minutes (FIG. 11C), or 60 minutes (FIG. 11D) in either TE buffer at pH 8.0 (left) or PBS (right).

The results show that permeabilization for 30 minutes generated the maximum cDNA signal generated from the mouse brain tissue. Additionally, while decrosslinking with PBS generally generated a weaker cDNA signal, the data demonstrate that PBS can be used as a decrosslinking buffer, although generally longer permeabilization times may be necessary.

Collectively, the data show that mRNA from FFPE samples was successfully captured under a variety of decrosslinking and permeabilization conditions and that cDNA derived from the captured mRNA with the addition of a TSO sequence was detected by Cy3 labeled TSO probes. That, coupled with successful amplification in FFPE biological samples, including mouse brain samples, soft tissue sarcoma samples (multiple ages), colon cancer, and ovarian carcinosarcoma samples, was able to identify experimental conditions that can be useful in generating cDNA from captured analytes, especially when the analytes are from fixed (e.g., FFPE) biological samples, in a spatial array.

TSO Hybridization Probe
SEQ ID NO: 1
AAGCAGTGGTATCAACGCAGAGTACATGGG

1-40. (canceled)

41. A method comprising:
(a) a biological sample on an array comprising a plurality of capture probes, wherein a capture probe of the plurality of capture probes comprises a capture domain that hybridizes to an analyte in the biological sample;
(b) extending a 3′ end of the capture probe using the analyte as a template to generate an extended capture probe;
(c) adding to a 3′ end of the extended capture probe a first homopolymeric sequence;
(d) contacting the extended capture probe with a template switching oligonucleotide comprising a strand of nucleic acid in a 5′ to 3′ direction: a primer binding sequence and a second homopolymeric sequence, where the second homopolymeric sequence hybridizes to the first homopolymeric sequence;
(e) extending the 3′ end of the extended capture probe using the template switching oligonucleotide as a template thereby generating an extended capture probe comprising a template-switching oligonucleotide sequence;
(f) removing the analyte and the template-switching oligonucleotide from the extended capture probe comprising the template-switching oligonucleotide sequence;
(g) hybridizing one or more labeled probes complementary to all or a portion of the template-switching oligonucleotide sequence of the extended capture probe comprising the template-switching oligonucleotide sequence; and
(h) detecting the one or more labeled probes hybridized to the template-switching oligonucleotide sequence of the extended capture probe comprising the template-switching oligonucleotide sequence.

42. The method of claim 41, wherein steps (a)-(h) are performed under a first set of conditions, and the method further comprises repeating steps (a)-(h) under a second set of conditions.

43. The method of claim 41, further comprising staining the biological sample, and optionally, wherein the staining comprises hematoxylin and eosin.

44. The method of claim 41, further comprising imaging the biological sample, wherein the imaging comprises one or more of light field microscopy, bright field microscopy, dark field microscopy, phase contrast microscopy, fluorescence microscopy, reflection microscopy, interference microscopy, and confocal microscopy.

45. The method of claim 41, wherein the capture probe of the plurality of capture probes further comprises a spatial barcode.

46. The method of claim 41, wherein the capture probe of the plurality of capture probes further comprises one or more functional domains, a unique molecular identifier, a cleavage domain, and combinations thereof.

47. The method of claim 41, wherein a labeled probe of the one or more labeled probes comprises a fluorescent label.

48. The method of claim 41, wherein adding to the 3′ end of the extended capture probe the first homopolymeric sequence in step (c) is performed using a terminal deoxynucleotidyl transferase.

49. The method of claim 41, wherein removing the analyte and the template-switching oligonucleotide from the extended capture probe comprising the template-switching oligonucleotide in step (f) comprises one or more washing steps.

50. The method of claim 49, wherein the one or more washing steps comprises potassium hydroxide.

51. The method of claim 41, wherein the array comprises one or more features selected from the group consisting of: a bead, a well, and a spot.

52. The method of claim 41, wherein the biological sample is a tissue sample or a tissue section.

53. The method of claim 52, wherein the tissue sample is a fixed tissue sample.

54. The method of claim 52, wherein the tissue section is a fixed tissue section.

55. The method of claim 54, wherein the fixed tissue section is a formalin-fixed paraffin-embedded tissue section.

56. The method of claim 41, wherein the analyte is DNA.

57. The method of claim 41, wherein the analyte is mRNA.

58. The method of claim 41, wherein the capture domain comprises a poly(T) sequence.

59. The method of claim 41, wherein the first homopolymeric sequence is a poly(C) sequence and the second homopolymeric sequence is a poly(G) sequence.

60. A method comprising:
(a) contacting a biological sample with an array comprising a plurality of capture probes, wherein a capture probe of the plurality of capture probes comprises a capture domain that hybridizes to an analyte in the biological sample;
(b) staining and imaging the biological sample;
(c) extending a 3′ end of the capture probe using the analyte as a template to generate an extended capture probe;
(d) adding to a 3′ end of the extended capture probe a first homopolymeric sequence;
(e) contacting the extended capture probe with a template-switching oligonucleotide comprising in a 5′ to 3′ direction: a primer binding sequence and a second homopolymeric sequence, wherein the second homopolymeric sequence hybridizes to the first homopolymeric sequence;
(f) extending the 3′ end of the extended capture probe using the template-switching oligonucleotide as a template thereby generating an extended capture probe comprising a template-switching oligonucleotide sequence;
(g) removing the analyte and the template-switching oligonucleotide from the extended capture probe comprising the template-switching oligonucleotide sequence;
(h) hybridizing one or more labeled probes comprising a sequence complementary to the template-switching oligonucleotide sequence, or a portion thereof, of the extended capture probe comprising the template-switching oligonucleotide sequence; and
(i) detecting the one or more labeled probes hybridized to the template-switching oligonucleotide sequence of the extended capture probe comprising the template-switching oligonucleotide sequence.


SEQUENCE LISTING (1 sequence)

SEQ ID NO: 1 - DNA, 30 bp, Artificial
  TSO Hybridization Probe
aagcagtggtatcaacgcagagtacatggg